(19) United States
(12) Patent Application Publication
(10) Pub. No.: US 2009/0233965 A1
(43) Pub. Date: Sep. 17, 2009

(54) Alkyl Esters Of Cyclic Amino Alcohols With Muscarinic M3 Receptor Antagonist Activity, Useful For Treating E.G. Chronic Bronchial Obstruction, Asthma And Overactive Bladder

(75) Inventors: Andrew Bailey, Leicestershire (GB); Antonio Mete, Leicestershire (GB); Garry Pairaudeau, Molndal (SE); Michael Stocks, Leicestershire (GB); Mark Wenlock, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/298,187

(22) PCT Filed: Apr. 23, 2007
(86) PCT No.: PCT/SE07/00389

§ 371 Date: Apr. 8, 2009

Related U.S. Application Data
(60) Provisional application No. 60/794,433, filed on Apr. 24, 2006.
(60) Provisional application No. 60/828,391, filed on Oct. 6, 2006.

(51) Int. Cl. A61K 31/445; C07D 211/00; C07D 409/12; C07D 401/12

(52) U.S. Cl. 514/318; 546/222; 514/327; 514/326; 546/213; 546/194

(57) ABSTRACT

The invention provides compounds of formula (I), wherein R¹, R², R³, R⁴, R⁵ and X are as defined in the specification, a process for their preparation, pharmaceutical compositions containing them, a process for preparing pharmaceutical compositions and their use in therapy (I).

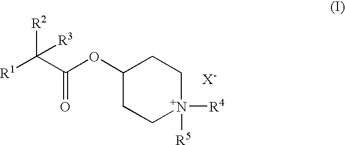

The present invention relates to substituted alkyl esters of cyclic amino alcohols, a process for their preparation, pharmaceutical compositions containing them, a process for preparing pharmaceutical compositions and their use in therapy.

Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue.

Parasympathetic nerves are the main pathway for reflex bronchoconstriction in human airways and mediate airway tone by releasing acetylcholine onto muscarinic receptors. Airway tone is increased in patients with respiratory disorders such as asthma and chronic is obstructive pulmonary disease (COPD), and for this reason muscarinic receptor anatgonists have been developed for use in treating airway diseases. Muscarinic receptor antagonsists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1, 223-229).

When used to treat respiratory disorders, muscarinic receptor antagonists are typically administered by inhalation. However, when administered by inhalation a significant proportion of the muscarinic receptor antagonist is often absorbed into the systemic circulation resulting in reported side effects such as dry mouth. Additionally, the majority of muscarinic antagonists have a relatively short duration of action requiring that they be administered several times a day. Such a multiple-daily dosing regime is not only inconvenient to the patient but also creates a significant risk of inadequate treatment due to patient non-compliance associated with the frequent repeat dosing schedule.

There therefore remains a need for novel compounds that are capable of blocking muscarinic receptors. In particular, a need exists for new muscarinic antagonists that have high potency and reduced systemic side effects when administered by inhalation.

Moreover, a need exists for new muscarinic antagonists that exhibit a long duration of action when dosed by inhalation, and which are amenable to either once or twice daily dosing.

In accordance with the present invention, there is provided a compound of formula (I):

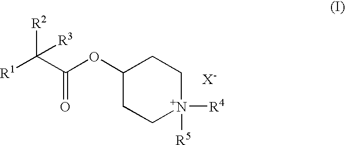

wherein:
R¹ represents phenyl, benzimidazolyl, benzthiazolyl, benzoxazolyl or a 5-6 membered heteroaromatic ring, each of which may be optionally substituted by one or more substituents independently selected from halogen, cyano, nitro, S(O)₀₋₂R⁶, NR⁷R⁸, S(O)NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₅ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³⁰S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
R⁵ represents hydrogen or C₁₋₆ alkyl;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, C₁₋₆ alkoxy, NH₂, NH(C₁₋₆ alkyl) and N(C₁₋₆ alkyl)₂;
or any of R⁷ and R⁸R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;
and X represents a pharmaceutically acceptable anion of a mono or polyvalent acid.

The compounds of formula (I) comprise an equivalent of an anion X associated with the positive charge on the quaternary nitrogen atom. The anion X may be any pharmaceutically acceptable anion of a mono or polyvalent (e.g. bivalent) acid. In an embodiment of the invention X may be an anion of a mineral acid, for example chloride, bromide, iodide, sulfate, nitrate or phosphate; or an anion of a suitable organic acid, for example acetate, maleate, fumarate, citrate, oxalate, succinate, tartrate, methanesulphonate or p-toluenesulphonate.

It will be understood that certain compounds of the present invention may exist in solvated, for example hydrated, as well as unsolvated forms. It is to be understood that the present invention encompasses all such solvated forms. Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

In the context of the present specification the term ‘Heteroaromatic’ denotes aromatic rings comprising at least one heteroatom selected from the group consisting of nitrogen, oxygen and sulfur. Examples of 5-6 membered heteroaromatic rings according to the present invention include thienyl, furanyl, pyridinyl, pyrimidinyl, pyrazinyl, pyridazinyl, thiazolyl, oxazolyl, oxadiazolyl, imidazolyl, isoxazolyl, isothiazolyl, pyrazolyl and triazolyl. The term ‘Aliphatic heterocyclic ring’ denotes non-aromatic rings comprising at least one heteroatom selected from the group consisting of nitrogen, oxygen and sulfur. Examples of 4-8 membered aliphatic heterocyclic rings according to the present invention include pyrrolidinyl, piperidinyl, piperazinyl, morpholinyl, homopiperazinyl, homopiperidinyl and azetidinyl.

Unless otherwise stated, in the context of the present specification alkyl groups and moieties may be straight or branched chain and include, for example, methyl, ethyl, n-propyl, iso-propyl, n-butyl, iso-butyl or tert-butyl. Cycloalkyl groups are monocyclic, for example cyclopropyl, cyclopentyl or cyclobutyl. Halogen is for example, fluorine, chlorine or bromine.

In the context of the present specification, where it is stated that a group may be optionally substituted with one or more substituents the group may be unsubstituted or substituted; when substituted the group will generally be substituted with one, two or three substituents.

In an embodiment of the invention, R¹ represents phenyl, pyridinyl or thienyl which phenyl, pyridinyl or thienyl may be optionally substituted by one or more substituents independently selected from halogen, cyano, hydroxyl, C₁₋₆ alkoxy, SC₁₋₄ alkyl, SO₂C₁₋₄ alkyl, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂.

In an embodiment of the invention, R¹ represents phenyl, pyridinyl or thienyl which phenyl, pyridinyl or thienyl may be optionally substituted by one or more substituents independently selected from halogen, cyano, hydroxyl, C₁₋₄ alkoxy, SC₁₋₄ alkyl, SO₂C₁₋₄ alkyl and C₁₋₄ alkyl.

In an embodiment of the invention, R¹ represents phenyl or pyridinyl, which phenyl or pyridinyl may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R¹ represents phenyl or pyridinyl which phenyl or pyridinyl may be optionally substituted by one or more substituents independently selected from halogen, hydroxyl, NH₂, NH(C₁₋₄ alkyl) and N(C₁₋₄ alkyl)₂.

In an embodiment of the invention, R¹ represents phenyl, which phenyl may be optionally substituted by one or more substituents independently selected from halogen, cyano, hydroxyl, C₁₋₆ alkoxy, SC₁₋₄ alkyl, SO₂C₁₋₄ alkyl, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R¹ represents phenyl which may be optionally substituted by one or more substituents independently selected from halogen, cyano hydroxyl, C₁₋₄ alkoxy, SC₁₋₄ alkyl, SO₂C₁₋₄ alkyl, NH₂, NH(C₁₋₄ alkyl) and N(C₁₋₄ alkyl)₂ and C₁₋₄ alkyl.

In an embodiment of the invention, R¹ represents phenyl, which phenyl may be optionally substituted by one or more substituents independently selected from halogen, cyano, hydroxyl, C₁₋₄ alkoxy, SC₁₋₄ alkyl, SO₂C₁₋₄ alkyl and C₁₋₄ alkyl.

In an embodiment of the invention, R¹ represents pyridinyl, which pyridinyl may be optionally substituted by one or more substituents independently selected from halogen, cyano, hydroxyl, C₁₋₆ alkoxy, SC₁₋₄ alkyl, SO₂C₁₋₄ alkyl, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R¹ represents pyridinyl which may be optionally substituted by one or more substituents independently selected from halogen, hydroxyl, NH₂, NH(C₁₋₄ alkyl) and N(C₁₋₄ alkyl)₂.

In an embodiment of the invention, R¹ represents thienyl, which thienyl may be optionally substituted by one or more substituents independently selected from halogen, cyano, hydroxyl, C₁₋₆ alkoxy, SC₁₋₄ alkyl, SO₂C₁₋₄ alkyl, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R¹ represents thienyl which may be optionally substituted by one or more substituents independently selected from halogen, hydroxyl, NH₂, NH(C₁₋₄ alkyl) and N(C₁₋₄ alkyl)₂.

In an embodiment of the invention, R² represents a C₃₋₅ cycloalkyl ring which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂.

In an embodiment of the invention, R² represents cyclopentyl which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R² represents cyclopentyl which may be optionally substituted by one or more substituents independently selected from halogen, hydroxyl, NH₂, NH(C₁₋₄ alkyl) and N(C₁₋₄ alkyl)₂.

In an embodiment of the invention, R² represents cyclopropyl which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R² represents cyclopropyl which may be optionally substituted by one or more substituents independently selected from halogen, hydroxyl, NH₂, NH(C₁₋₄ alkyl) and N(C₁₋₄ alkyl)₂.

In an embodiment of the invention, R² represents cyclobutyl which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R² represents cyclobutyl which may be optionally substituted by one or more substituents independently selected from halogen, hydroxyl, NH₂, NH(C₁₋₄ alkyl) and N(C₁₋₄ alkyl)₂.

In an embodiment of the invention, R³ represents methyl, ethyl or n-propyl. In a further aspect of this embodiment, R³ represents methyl.

In an embodiment of the invention, R⁴ represents hydrogen, methyl, ethyl or n-propyl. In a further aspect of this embodiment, R⁴ represents methyl.

In an embodiment of the invention, R⁵ represents methyl, ethyl or n-propyl. In a further aspect of this embodiment, R⁵ represents methyl.

In an embodiment of the invention, R⁴ and R⁵ each independently represent methyl or ethyl.

In an embodiment of the invention, X represents chloride, bromide, iodide or fumarate. In a further aspect of this embodiment, X represents bromide or iodide.

In an embodiment of the invention, R¹ represents phenyl, pyridinyl or thienyl which phenyl, pyridinyl or thienyl may be optionally substituted by one or more substituents independently selected from halogen, cyano, hydroxyl, C₁₋₆ alkoxy, SC₁₋₄ alkyl, SO₂C₁₋₄ alkyl, NH₂, NH(C₁₋₄ alkyl), N(C₁₋₄ alkyl)₂ and C₁₋₄ alkoxy, R² represents a C₃₋₅ cycloalkyl ring which may be optionally substituted by one or more substituents independently selected from halogen, hydroxyl, NH₂, NH(C₁₋₄ alkyl) and N(C₁₋₄ alkyl)₂; R³ represents methyl; R⁴ and R⁵ each independently represent methyl or ethyl; and X represents a pharmaceutically acceptable anion of a mono or polyvalent acid. In a further aspect of this embodiment, X represents chloride, bromide, iodide or fumarate.

The compounds of the invention have a chiral center located at the carbon atom to which each of R¹, R² and R³ are bonded (the 2′ position). The present invention encompasses all optical isomers of the compounds of formula (I) and mixtures thereof including racemates.

In an embodiment of the invention, the chiral center located at the 2′ position has an R configuration. In a further embodiment of the invention, the chiral center located at the 2′ position has an S configuration.

In a further embodiment, the present invention provides an optically pure compound of formula (I). In the context of the present specification, the term optically pure is defined in terms of enantiomeric excess (e.e.), which is calculated from the ratio of the difference between the amounts of the respective enantiomers present and the sum of these amounts, expressed as a percentage. To illustrate, a preparation containing 95% of one enantiomer and 5% of another enantiomer has an enantiomeric excess (e.e.) of 90% [i.e. (95-5)/(95+5)×100]. Optically pure compounds according to the present invention have an e.e. of at least 90%. In an embodiment of the invention, optically pure compounds have an e.e. of at least 95%. In a further embodiment of the invention, optically pure compounds have an e.e. of at least 98%.

In an embodiment of the invention, there is provided an optically pure compound of formula (I) having a 2′R configuration.

In an embodiment of the invention, there is provided an optically pure compound of formula (I) having a 2′S configuration.

In a further embodiment the present invention provides a compound of formula (IB);

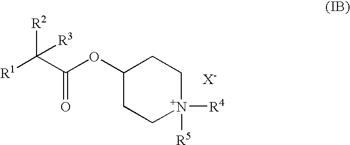

wherein:
R¹ represents phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;

R² represents a C₃₋₅ cycloalkyl ring, which cycloalkyl ring may be optionally substituted by one or more substituents independently selected from halogen, S(O)₀₋₂R²⁴, NR²⁵R²⁶, S(O)NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected is from halogen, hydroxyl, C₁₋₆ alkoxy, NH₂, NH(C₁₋₆ alkyl) and N(C₁₋₆ alkyl)₂;

R³ represents C₁₋₆ alkyl;
R⁴ represents hydrogen or C₁₋₆ alkyl;
R⁵ represents hydrogen or C₁₋₆ alkyl;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;

R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, C₁₋₆ alkoxy, NH₂, NH(C₁₋₆ alkyl) and N(C₁₋₆ alkyl)₂; or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;

and X represents a pharmaceutically acceptable anion of a mono or polyvalent acid.

In an embodiment of the invention, in formula (IB) R¹ represents phenyl or pyridinyl, which phenyl or pyridinyl may be optionally substituted by one or more substituents independently selected from halogen, hydroxyl, NH₂, NH(C₁₋₄ alkyl) and N(C₁₋₄ alkyl)₂; R² represents a C₃₋₅ cycloalkyl ring which may be optionally substituted by one or more substituents independently selected from halogen, hydroxyl, NH₂, NH(C₁₋₄ alkyl) and is N(C₁₋₄ alkyl)₂; R³ represents methyl; R⁴ and R⁵ each independently represent methyl or ethyl; and X represents a pharmaceutically acceptable anion of a mono or polyvalent acid. In a further aspect of this embodiment, X represents chloride, bromide, iodide or fumarate.

In an embodiment of the invention, the compound of formula (I) is selected from:
4-{[(2-Cyclopentyl-2-phenylpropanoyl]oxy}-1,1-dimethylpiperidinium X,
4-[(2-Cyclopropyl-2-phenylpropanoyl)oxy]-1,1-dimethylpiperidinium X,
4-[(2-Cyclopentyl-2-pyridin-3-ylpropanoyl)oxy]-1,1-dimethylpiperidinium X,
4-{[2-Cyclopentyl-2-(4-hydroxyphenyl)propanoyl]oxy}-1,1-dimethylpiperidinium X,
4-[(2-Cyclobutyl-2-phenylpropanoyl)oxy]-1,1-dimethylpiperidinium X,
4-{[2-Cyclopentyl-2-(4-methoxyphenyl)propanoyl]oxy}-1,1-dimethylpiperidinium X,
4-{[2-Cyclopentyl-2-(2-thienyl)propanoyl]oxy}-1,1-dimethylpiperidinium X,
4-{[2-Cyclopentyl-2-(5-methyl-2-thienyl)propanoyl]oxy}-1,1-dimethylpiperidinium X,
4-{[2-(3-Bromophenyl)-2-cyclopentylpropanoyl]oxy}-1,1-dimethylpiperidinium X,
4-{[2-(4-Bromophenyl)-2-cyclopentylpropanoyl]oxy}-1,1-dimethylpiperidinium X,
4-{[2-(4-Cyanophenyl)-2-cyclopentylpropanoyl]oxy}-1,1-dimethylpiperidinium X,
4-{[2-(3-Cyanophenyl)-2-cyclopentylpropanoyl]oxy}-1,1-dimethylpiperidinium X,
4-{[2-(3-Methylthiophenyl)-2-cyclopentylpropanoyl]oxy}-1,1-dimethylpiperidinium X,
4-{[2-(4-Methylthiophenyl)-2-cyclopentylpropanoyl]oxy}-1,1-dimethylpiperidinium X,
4-{[2-(4-Methylsulfonylphenyl)-2-cyclopentylpropanoyl]oxy}-1,1-dimethylpiperidinium
4-{[2-(3-Methylsulfonylphenyl)-2-cyclopentylpropanoyl]oxy}-1,1-dimethylpiperidinium X,
4-{[2-(4-Fluorophenyl)-2-cyclopentylpropanoyl]oxy}-1,1-dimethylpiperidinium X,
4-{[2-(4-Chlorophenyl)-2-cyclopentylpropanoyl]oxy}-1,1-dimethylpiperidinium X,
4-{[2-(3-Fluorophenyl)-2-cyclopentylpropanoyl]oxy}-1,1-dimethylpiperidinium X, and
4-{[2-(5-Chloro-2-thienyl)-2-cyclobutylpropanoyl]oxy}-1,1-dimethylpiperidinium X
   wherein X represents a pharmaceutically acceptable anion of a mono or polyvalent acid. Pharmaceutically acceptable anions according to this embodiment include chloride, bromide, iodide and fumarate. It will be understood that the compounds of this embodiment may be in the form of either the S or R isomer, or mixtures thereof including racemates.

In a further embodiment, the compound of formula (I) is selected from:
4-{[(2S)-2-Cyclopentyl-2-phenylpropanoyl]oxy}-1,1-dimethylpiperidinium X,
4-{[(2R)-2-Cyclopentyl-2-phenylpropanoyl]oxy}-1,1-dimethylpiperidinium X,
4-{[(2S)-Cyclopentyl-2-(2-thienyl)propanoyl]oxy}-1,1-dimethylpiperidinium X,
4-{[(2R)-Cyclopentyl-2-(2-thienyl)propanoyl]oxy}-1,1-dimethylpiperidinium X,
4-{[(2S)-Cyclopentyl-2-(5-methyl-2-thienyl)propanoyl]oxy}-1,1-dimethylpiperidinium X,
4-{[(2R)-Cyclopentyl-2-(5-methyl-2-thienyl)propanoyl]oxy}-1,1-dimethylpiperidinium X,
4-{[(2S)-(5-Chloro-2-thienyl)-2-cyclobutylpropanoyl]oxy}-1,1-dimethylpiperidinium X,
4-{[(2R)-(5-Chloro-2-thienyl)-2-cyclobutylpropanoyl]oxy}-1,1-dimethylpiperidinium,
4-[(2S)-Cyclopropyl-2-phenylpropanoyl)oxy]-1,1-dimethylpiperidinium X, and
4-[(2R)-Cyclopropyl-2-phenylpropanoyl)oxy]-1,1-dimethylpiperidinium X
   wherein X represents a pharmaceutically acceptable anion of a mono or polyvalent acid. Pharmaceutically acceptable anions according to this embodiment include chloride, bromide, iodide and fumarate.

In a further aspect, the present invention provides a process for the preparation of compounds of formula (I), which comprises reacting a compound of formula (IV) wherein R¹, R² and R³ are as defined in formula (I)

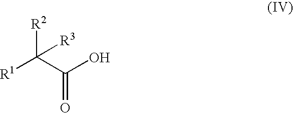

or a C₁₋₆alkyl ester, acid anhydride or acid halide-thereof, with a compound of formula (V), wherein R⁴ is as defined in formula (I)

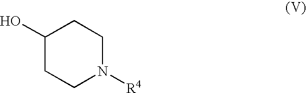

to yield a compound of formula (II)

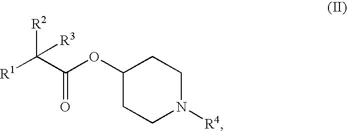

and subsequently reacting (II) with a compound of formula R⁵—Y (III), wherein Y is a leaving group (e.g. halogen) and R⁵ is as defined in formula (I),
and optionally carrying out one or more of the following:
converting the compound to a further compound of formula (I),
forming a pharmaceutically acceptable salt with an anion of a mono or polyvalent acid.

The reaction of compounds (IV) and (V) may be conveniently conducted in the presence of a suitable solvent such as toluene or dichloromethane at a temperature in the range of 0 to 100° C. In one embodiment of the invention, compound (IV) may conveniently take the form of an acid halide (e.g. chloride) as may be prepared by reacting the acid with a suitable reagent (e.g. thionyl chloride or oxalyl chloride) in a suitable solvent such as dichloromethane or toluene, at a temperature in the range of 0 to 100° C.

The reaction of compounds (II) and (III) may be conveniently conducted in the presence of a suitable solvent such as dichloromethane or acetonitrile at a temperature in the range of 0 to 100° C.

Compounds of formula (IV) may be conveniently prepared by reaction of a compound of formula

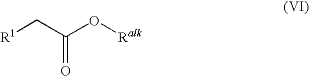

wherein R^{alk} is methyl, ethyl or OR^{alk} is a group of formula (V), with a base such as lithium diisopropylamide or sodium hexamethyldisilazide in a suitable solvent such as tetrahydrofuran or diethyl ether followed by treatment with a group of formula R²-LG wherein R² is as defined in (I) and LG is a leaving group such as bromide, iodide, methanesulfonate or tosylsulfonate. Subsequent reaction of the resulting compound R¹R²CHCO₂R^{alk} (VII) with a base such as lithium diisopropylamide or sodium hexamethyldisilazide in a suitable solvent such as tetrahydrofuran or diethyl ether followed by treatment with a group of formula R³-LG wherein R³ and LG are as defined above, yields compounds of formula (IV)

Compounds of formula (IV) may alternatively be prepared by treatment of a compound of formula

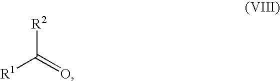

with trimethylsulfonium iodide ylide (prepared by treatment of the trimethylsulfonium iodide with a base such as sodium hydride in a solvent such as DMSO). Subsequent treatment with an acid or Lewis acid such as Irridium trichloride in a solvent such as tetrahydrofuran, followed by oxidation with a reagent such as sodium chlorite and generation of the ester from the resulting acid by treatment with trimethylsilyldiazomethane in a solvent mixture such as methanol/tetrahydrofuran or treatment with acid such as tolylsulfonic acid in methanol to give a compound of formula (VII) as defined above.

Compounds of formula (IV) may alternatively be prepared by treatment of a compound of formula (VIII), with methoxymethyl(triphenylphosphonium) chloride with a suitable base such as butyllithium, followed by treatment of the resultant enol ether with an acid such as formic acid in a suitable solvent such as tetrahydrofuran followed by oxidation with a reagent such as sodium chlorite and generation of the ester from the resulting acid by treatment with trimethylsilyldiazomethane in a solvent mixture such as methanol/tetrahydrofuran or treatment with acid such as tolylsulfonic acid in methanol to give a compound of formula (VII) as defined above.

It will be appreciated by those skilled in the art that in the processes of the present invention certain functional groups such as hydroxyl, carboxyl or amino groups in the starting reagents or intermediate compounds may need to be protected by protecting groups. Thus, the preparation of the compounds of formula (I) may involve at a certain stage the removal of one or more protecting groups. The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994).

Compounds of formula (IV) may be prepared using methods described, or analogous to those described, in the art. For example, the preparation of α-cycloalkyl-α-phenylpropionic acids is described in Zhumal Obshchei Khimii, (1964), 34 (5), 1618-1621.

Compounds of formula (V) can be obtained commercially or can be prepared using methods described in the literature.

Optically pure compounds of formula (I) may, for example, be prepared by reacting an optically pure compound of formula (IV) with a compound of formula (V), to give an ester of formula (II), and subsequently reacting (II) with a compound of formula (III). Optically pure compounds of formula (IV) may be prepared by reacting the corresponding racemic acid, or C₁₋₆alkyl ester, acid anhydride or acid halide thereof, with a suitable auxiliary compound, (e.g. (R)-4-benzyl-2-oxazolidinone) or (2R)-bornane-10,2-sultam), separating the resulting mixture of diastereomeric esters (e.g. by chromatography), and subsequently removing the auxiliary compound to yield an optically pure acid of formula (IV).

Compounds of formula (II) have not been prepared previously. Moreover, these non-quaternised compounds also display activity as anticholinergic agents and are of interest for use in treating conditions of the urinary tract, such as overactive bladder. Accordingly, the present invention further provides a compound of formula (II)

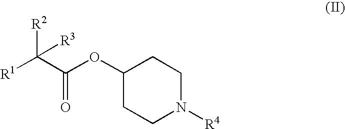

wherein,
R¹ represents phenyl, benzimidazolyl, benzthiazolyl, benzoxazolyl or a 5-6 membered heteroaromatic ring, each of which may be optionally substituted by one or more substituents independently selected from halogen, cyano, nitro, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₅ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
R⁶, R¹³, R¹⁵R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, C₁₋₆ alkoxy, NH₂, NH(C₁₋₆ alkyl) and N(C₁₋₆ alkyl)₂; or any of R⁷ and R⁸, R⁹ and R¹⁰, R¹¹ and R¹², R²¹ and R²², R²⁵ and R²⁶, R²⁷ and R²⁵, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl.

For compounds of formula (HI), embodiments of the invention include those wherein each of R¹, R², R³ and R⁴ are as defined herein above in embodiments of the invention concerning compounds of formula (I).

In an embodiment of the invention the compounds of formula (II) are selected from
1-Methylpiperidin-4-yl 2-cyclopentyl-2-phenylpropanoate,
1-Methylpiperidin-4-yl 2-cyclopropyl-2-phenylpropanoate,
1-Methylpiperidin-4-yl 2-cyclopentyl-2-pyridin-3-ylpropanoate,
1-Methylpiperidin-4-yl 2-cyclopentyl-2-(4-hydroxyphenyl)propanoate,
1-Methylpiperidin-4-yl 2-cyclobutyl-2-phenylpropanoate,
1-Methylpiperidin-4-yl 2-cyclopentyl-2-(2-thienyl)propanoate,
1-Methylpiperidin-4-yl 2-cyclopentyl-2-(5-methyl-2-thienyl)propanoate,
1-Methylpiperidin-4-yl 2-(3-bromophenyl)-2-cyclopentylpropanoate,
1-Methylpiperidin-4-yl 2-(4-bromophenyl)-2-cyclopentylpropanoate,
1-Methylpiperidin-4-yl 2-(4-cyanophenyl)-2-cyclopentylpropanoate,
1-Methylpiperidin-4-yl 2-(3-cyanophenyl)-2-cyclopentylpropanoate,
1-Methylpiperidin-4-yl 2-(3-methylthiophenyl)-2-cyclopentylpropanoate,
1-Methylpiperidin-4-yl 2-(4-methylthiophenyl)-2-cyclopentylpropanoate,
1-Methylpiperidin-4-yl 2-(4-methylsulfonylphenyl)-2-cyclopentylpropanoate,
1-Methylpiperidin-4-yl 2-(3-methylsulfonylphenyl)-2-cyclopentylpropanoate,
1-Methylpiperidin-4-yl 2-(4-chlorophenyl)-2-cyclopentylpropanoate,
1-Methylpiperidin-4-yl 2-(4-fluorophenyl)-2-cyclopentylpropanoate,
1-Methylpiperidin-4-yl 2-(3-fluorophenyl)-2-cyclopentylpropanoate,
1-Methylpiperidin-4-yl 2-(5-chloro-2-thienyl)-2-cyclobutylpropanoate.

It will be understood that the compounds of this embodiment may be in the form of either the S or R isomer, or mixtures thereof including racemates.

In another embodiment of the invention the compounds of formula (II) are selected from
1-Methylpiperidin-4-yl (2S)-2-cyclopentyl-2-phenylpropanoate,
1-Methylpiperidin-4-yl (2R)-2-cyclopentyl-2-phenylpropanoate,
1-Methylpiperidin-4-yl (2S)-2-cyclopentyl-2-(2-thienyl)propanoate,
1-Methylpiperidin-4-yl (2R)-2-cyclopentyl-2-(2-thienyl)propanoate,
1-Methylpiperidin-4-yl (25)-2-cyclopentyl-2-(5-methyl-2-thienyl)propanoate,
1-Methylpiperidin-4-yl (2R)-2-cyclopentyl-2-(5-methyl-2-thienyl)propanoate,
1-Methylpiperidin-4-yl (28)-2-cyclopropyl-2-phenylpropanoate and
1-Methylpiperidin-4-yl (2R)-2-cyclopropyl-2-phenylpropanoate.

In a further embodiment, the present invention provides an optically pure compound of formula (II), wherein optically pure is as previously defined herein before with respect to compounds of formula (I).

The compounds of the invention have activity as pharmaceuticals, in particular as anticholinergic agents including muscarinic receptor (M1, M2, and M3) antagonists, in particular M3 antagonists. Diseases and conditions which may be treated with the compounds include:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention further provides a compound of formula (I), as hereinbefore defined for use in therapy.

In another aspect, the invention provides the use of a compound of formula (I), as hereinbefore defined, in the manufacture of a medicament for use in therapy.

In the context of the present specification, the term “therapy” also includes “prophylaxis” unless there are specific indications to the contrary. The terms “therapeutic” and “therapeutically” should be construed accordingly.

A further aspect of the invention provides a method of treating a disease state in a mammal suffering from, or at risk of, said disease, which comprises administering to a mammal in need of such treatment a therapeutically effective amount of a compound of formula (I) as hereinbefore defined.

The present invention also provides the use of a compound of formula (I) as hereinbefore defined, in the manufacture of a medicament for use in the treatment of chronic obstructive pulmonary disease (COPD) (such as irreversible COPD).

The present invention also provides the use of a compound of formula (I) as hereinbefore defined, in the manufacture of a medicament for use in the treatment of asthma.

The present invention further provides a method of treating chronic obstructive pulmonary disease (COPD) (such as irreversible COPD), in a warm-blooded animal, such as man, which comprises administering to a mammal in need of such treatment an effective amount of a compound of formula (I) as hereinbefore defined.

The present invention further provides a method of treating asthma in a warm-blooded animal, such as man, which comprises administering to a mammal in need of such treatment an effective amount of a compound of formula (I) as hereinbefore defined.

In order to use a compound of the invention for the therapeutic treatment of a warm-blooded animal, such as man, said ingredient is normally formulated in accordance with standard pharmaceutical practice as a pharmaceutical composition.

Therefore in another aspect the present invention provides a pharmaceutical composition that comprises a compound of the invention as hereinbefore defined and a pharmaceutically acceptable adjuvant, diluent or carrier. In a further aspect the present invention provides a process for the preparation of said composition, which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule, which contains between 0.1 mg and 1 g of active ingredient.

In another aspect a pharmaceutical composition of the invention is one suitable for intravenous, subcutaneous or intramuscular injection. Each patient may receive, for example, an intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose, which is approximately equivalent to the daily parenteral dose, the composition being administered 1 to 4 times per day.

Another suitable pharmaceutical composition of this invention is one suitable for inhaled administration, inhalation being a particularly useful method for administering the compounds of the invention when treating respiratory diseases such as chronic obstructive pulmonary disease (COPD) or asthma. When administered by inhalation the compounds of formula (I) may be used effectively at doses in the μg range, for example 0.1 to 500 μg, 0.1 to 50 μg, 0.1 to 40 μg, 0.1 to 30 μg, 0.1 to 20 μg, 0.1 to 0 μg, 5 to 10 μg, 5 to 50 μg, 5 to 40 μg, 5 to 30 μg, 5 to 20 μg, 5 to 10 μg, 10 to 50 μg, 10 to 40 μg 10 to 30 μg, or 10 to 20 μg of active ingredient.

In an embodiment of the invention, there is provided a pharmaceutical composition comprising a compound of the invention as hereinbefore defined, in association with a pharmaceutically acceptable adjuvant, diluent or carrier, which is formulated for inhaled administration.

When administered by inhalation, metered dose inhaler devices may be used to administer the active ingredient, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations.

Dry powder inhalers may be used to administer the active ingredient, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The invention further relates to combination therapies wherein a compound of the invention or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention and a receptor antagonist for leukotrienes (LT) B4, LTC4, LTD4, and LTE4 selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a proton pump inhibitor (such as omeprazole) or a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention still further relates to the combination of a compound of the invention and a beta-adrenoceptor agonist (including beta receptor subtypes 1-4) such as isoprenaline, salbutamol, formoterol, salmeterol, terbutaline, orciprenaline, bitolterol mesylate, pirbuterol, or indacaterol or a chiral enantiomer thereof.

The present invention further relates to the combination of a compound of the invention and a chromone, such as sodium cromoglycate or nedocromil sodium.

The present invention still further relates to the combination of a compound of the invention with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention with an agent that modulates a nuclear hormone receptor such as PPARs.

The present invention still further relates to the combination of a compound of the invention together with an immunoglobulin (Ig) or Ig preparation or an antagonist or antibody modulating Ig function such as anti-IgE (for example omalizumab).

The present invention further relates to the combination of a compound of the invention and another systemic or topically-applied anti-inflammatory agent, such as thalidomide or a derivative thereof, a retinoid, dithranol or calcipotriol.

The present invention still further relates to the combination of a compound of the invention and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention and an agent for the treatment of acute or chronic pain, such as a centrally or peripherally-acting analgesic (for example an opioid or derivative thereof), carbamazepine, phenyloin, sodium valproate, amitryptyline or other anti-depressant agent-s, paracetamol, or a non-steroidal anti-inflammatory agent.

The present invention further relates to the combination of a compound of the invention together with a parenterally or topically-applied (including inhaled) local anaesthetic agent such as lignocaine or a derivative thereof.

A compound of the present invention can also be used in combination with an anti-osteoporosis agent including a hormonal agent such as raloxifene, or a biphosphonate such as alendronate.

The present invention still further relates to the combination of a compound of the invention together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B1.- or B2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK1 or NK3 receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; or (xxvii) inhibitor of transcription factor activation such as NFkB, API, or STATS.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafar, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a further embodiment the present invention provides a pharmaceutical product comprising, in combination, a first active ingredient which is a compound of formula (I), or a pharmaceutically acceptable salt thereof, as hereinbefore described, and at least one further active ingredient selected from:—
a phosphodiesterase inhibitor
a β2. adrenoceptor agonist
a modulator of chemokine receptor function
an inhibitor of kinase function
a protease inhibitor
a steroidal glucocorticoid receptor agonist
a non-steroidal glucocorticoid receptor agonist.

The pharmaceutical product according to this embodiment may, for example, be a pharmaceutical composition comprising the first and further active ingredients in admixture. Alternatively, the pharmaceutical product may, for example, comprise the first and further active ingredients in separate pharmaceutical preparations suitable for simultaneous, sequential or separate administration to a patient in need thereof. The pharmaceutical product of this embodiment is of particular use in treating respiratory diseases such as asthma, COPD or rhinitis.

Examples of a phosphodiesterase inhibitor that may be used in the pharmaceutical product according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044)
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (Cilomilast)
8-amino-1,3-bis(cyclopropylmethyl)xanthine (Cipamfylline)
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-yl methanesulphonate (Lirimilast),
(−)-(R)-5-(4-methoxy-3-propoxyphenyl)-5-methyloxazolidin-2-one (Mesopram),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine), 3-(cyclopropylmethoxy)-N-(3,5-dichloro-4-pyridyl)-4-(difluoromethoxy)benzamide (Roflumilast),
the N-oxide of Roflumilast,
5,6-diethoxybenzo[b]thiophene-2-carboxylic acid (Tibenelast)
2,3,6,7-tetrahydro-2-(mesitylimino)-9,10-dimethoxy-3-methyl-4H-pyrimido[6,1-a]isoquinolin-4-one (trequinsin) and
3-[[3-(cyclopentyloxy)-4-methoxyphenyl]-methyl]-N-ethyl-8-(1-methylethyl)-3H-purine-6-amine (V-11294A).

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonists, for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R-(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921, in US 2005/222144, compounds GSK 159797, GSK 159802, GSK 597901, GSK 642444 and GSK 678007.

Examples of a modulator of chemokine receptor function that may be used in the pharmaceutical product according to this embodiment include a CCR1 receptor antagonist.

Examples of an inhibitor of kinase function that may be used in the pharmaceutical product according to this embodiment include a p38 kinase inhibitor and an IKK inhibitor.

Examples of a protease inhibitor that may be used in the pharmaceutical product according to this embodiment include an inhibitor of neutrophil elastase or an inhibitor of MMP12.

Examples of a steroidal glucocorticoid receptor agonist that may be used in the pharmaceutical product according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of a modulator of a non-steroidal glucocorticoid receptor agonist that may be used in the pharmaceutical product according to this embodiment include those described in WO2006/046916

The present invention will now be illustrated with the following non-limiting Examples.

In the examples the NMR spectra were measured on a Varian Unity Inova spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% ammonium acetate: acetonitrile as the eluent. SCX and NH₂ resin were obtained from Varian Incorporated.

### EXAMPLE 1

### 4-{[(2S)-2-Cyclopentyl-2-phenylpropanoyl]oxy}-1,1-dimethylpiperidinium iodide

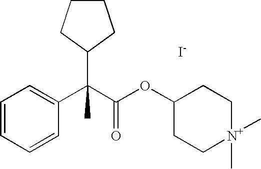

### a) Methyl cyclopentyl(phenyl)acetate

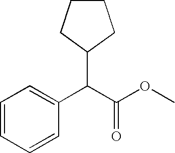

A solution of α-phenylcyclopentaneacetic acid (10 g) in methanol (200 mL) was treated with chlorotrimethylsilane (20 mL) and the mixture heated at reflux for 3 hours. The solvent was removed under reduced pressure and the residue was purified by chromatography on a silica column, eluting with 25% diethylether in iso-hexane to yield the sub-titled compound (10.5 g) as an oil.

¹H NMR (400 MHz, CDCl₃) δ 7.35-7.22 (m, 5H), 3.64 (s, 3H), 3.28 (d, 1H), 2.60-2.50 (m, 1H), 1.95-1.85 (m, 1H), 1.70-1.40 (m, 5H), 1.30-1.20 (m, 1H), 1.03-0.97 (m, 1H).

### b) Methyl 2-cyclopentyl-2-phenylpropanoate

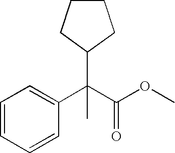

A 1.8 molar solution of lithium diisopropylamide in heptane/tetrahydrofuran/ethylbenzene (40.1 mL) was added to anhydrous tetrahydrofuran (60 mL) and stirred at −78° C. under nitrogen. To this mixture was then added, dropwise over 25 minutes, a solution of methyl cyclopentyl(phenyl)acetate (Example 1a), 10.5 g) in anhydrous tetrahydrofuran (30 mL). The reaction mixture was stirred at −78° C. for 15 minutes and then allowed to warm to −30° C. A solution of iodomethane (13.68 g) in anhydrous tetrahydrofuran (15 mL) was then added over a period of 10 minutes. The mixture was stirred at 0° C. for 1 hour and then partitioned between ethyl acetate and saturated aqueous ammonium chloride solution. The organic layer was separated, dried over anhydrous magnesium sulphate, filtered and the solvent removed under reduced pressure to yield the sub-titled compound (10.4 g) as an oil.

¹H NMR (400 MHz, CDCl₃) δ 7.36-7.19 (m, 5H), 3.64 (s, 3H), 2.83-2.76 (m, 1H), 1.77-1.69 (m, 1H), 1.60-1.30 (m, 9H), 0.95-0.85 (m, 1H).

### c) 2-Cyclopentyl-2-phenyl-propionic acid

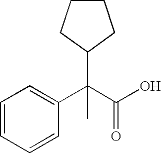

A mixture of methyl 2-cyclopentyl-2-phenylpropanoate (Example 1b), 10.4 g) in methanol (150 mL) and aqueous sodium hydroxide solution (2M, 100 mL) was refluxed for 18 hours. The methanol was evaporated under reduced pressure and the residue partitioned between water and diethyl ether, the aqueous layer was separated and cooled with ice. The aqueous solution was acidified with concentrated hydrochloric acid and the resultant precipitate extracted into ethyl acetate. The organic layer was then washed with brine, separated, dried over anhydrous magnesium sulphate and the solvent removed under reduced pressure to yield the sub-titled compound (7.7 g).

m/e 217 (M−H)⁺

### d) (4R)-4-Benzyl-3-[(2S)-2-cyclopentyl-2-phenylpropanoyl]-1,3-oxazolidin-2-one

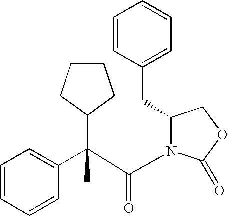

A solution of 2-cyclopentyl-2-phenyl-propionic acid prepared as described in Example 1c (4.93 g) in toluene (100 mL) was treated with thionyl chloride (30 mL) and the resulting mixture heated at 100° C. for 2 hours. The solvent was removed under reduced pressure and the residue azeotroped three times with toluene. The residue was dissolved in anhydrous tetrahydrofuran (20 mL) and added in one portion to a solution of (R)-4-benzyl-2-oxazolidinone (4.00 g) in anhydrous tetrahydrofuran (100 mL) at −78° C., which had been pre-treated at −78° C. with a 1.6 molar solution of n-butyllithium in hexanes (14.1 mL). The reaction mixture was stirred at −78° C. for 30 minutes and then at room temperature for 1 hour. At the end of this time the mixture was partitioned between ethyl acetate and saturated aqueous ammonium chloride solution, the organic layer was dried and evaporated under reduced pressure. Purification was by flash chromatography on a silica column, eluting with 10% tetrahydrofuran in iso-hexane to yield the sub-titled compound (3.3 g).

¹H NMR (400 MHz, DMSO-D₆) δ 7.32-7.24 (m, 7H), 7.18-7.12 (m, 3H), 4.72-4.70 (m, 1H), 4.20 (t, 1H), 4.01 (q, 1H), 3.20 (q, 1H), 2.81-2.74 (m, 2H), 1.78-1.72 (m, 1H), 1.59 (s, 3H), 1.49-1.17 (m, 7H).

### e) (S)-2-Cyclopentyl-2-phenyl-propionic acid

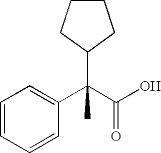

To a solution of (4R)-4-benzyl-3-[(2S)-2-cyclopentyl-2-phenylpropanoyl]-1,3-oxazolidin-2-one (Example 1d), 3.25 g) in a mixture of tetrahydrofuran (80 mL) and water (20 mL) stirred at 0° C. under nitrogen was added, dropwise a 35 wt. % solution of hydrogen peroxide in water (3.01 mL). The mixture was then treated dropwise with a solution of lithium hydroxide monohydrate (578 mg) in water (7 mL) followed by stirring for 4 hours at room temperature. At the end of this time the reaction mixture was cooled in an ice bath and treated with a solution of sodium metabisulphite (4 g) in water (30 mL). The mixture was evaporated under reduced pressure to remove the tetrahydrofuran and the residue was partitioned between diethyl ether and excess dilute aqueous sodium hydroxide. The aqueous layer was cooled and acidified by the dropwise addition of concentrated hydrochloric acid, the resultant precipitate was extracted into ethyl acetate, the ethyl acetate layer was washed with brine, dried over anhydrous magnesium sulphate and evaporated under reduced pressure. The crude product was purified by flash chromatography on a silica column, eluting with 20% ethyl acetate in iso-hexane to yield the sub-titled compound (1.7 g).

m/e 217.7 (M−H)⁺

¹H NMR (400 MHz, CDCl₃) δ 7.42-7.39 (m, 2H), 7.32 (t, 2H), 7.26-7.22 (m, 1H), 2.84-2.79 (m, 1H), 1.80-1.77 (m, 1H), 1.59-1.38 (m, 9H), 1.14-1.11 (m, 1H).

### f) 1-Methylpiperidin-4-yl (2S)-2-cyclopentyl-2-phenylpropanoate

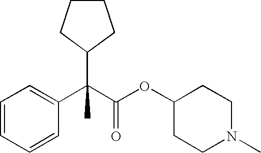

A solution of (2S)-2-cyclopentyl-2-phenylpropanoic acid (Example 1e), 249 mg) in toluene (20 mL) was treated with thionyl chloride (5 mL) and the resultant mixture heated at 100° C. for 2 hours. The solvent was removed under reduced pressure and the residue azeotroped three times with toluene. The residue was dissolved in dichloromethane (3 mL) and was treated with 4-hydroxy-1-methylpiperidine (394 mg) and the reaction mixture was heated at 40° C. for 18 hours. The reaction mixture was partitioned between dichloromethane and saturated aqueous sodium bicarbonate solution, the organic layer was dried over anhydrous magnesium sulphate and evaporated under reduced pressure. The crude product was purified by flash chromatography on a silica column, eluting with 1% triethylamine in ethyl acetate/iso-hexane (1/1) to yield the sub-titled compound (100 mg).

m/e 316 (M+H)⁺

### g) 4-{[(2S)-2-Cyclopentyl-2-phenylpropanoyl]oxy}-1,1-dimethylpiperidinium iodide

A solution of 1-methylpiperidin-4-yl (2S)-2-cyclopentyl-2-phenylpropanoate (Example 1f), 100 mg) in acetonitrile (1 mL) was treated with a solution of iodomethane (47 mg) in dichloromethane (0.5 mL). The mixture was allowed to stand at room temperature for 40 minutes. At the end of this time the solvents were evaporated under reduced pressure and the residue triturated with diethyl ether to yield the titled compound as a white solid (106 mg).

m/e 330.3 (M)⁺

¹H NMR (400 MHz, DMSO-D₆) δ 7.39-7.32 (m, 4H), 7.26-7.22 (m, 1H), 4.90-4.87 (m, 1H), 3.38-3.35 (m, 2H), 3.16-3.10 (m, 2H), 3.05 (s, 6H), 2.86-2.82 (m, 1H), 2.10-2.05 (m, 2H), 1.80-1.63 (m, 3H), 1.54-1.33 (m, 9H), 1.12-1.07 (m, 1H).

### EXAMPLE 2

### 4-{[(2R)-2-Cyclopentyl-2-phenylpropanoyl]oxy}-1,1-dimethylpiperidinium iodide

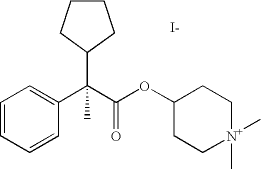

### a) (4R)-4-Benzyl-3-[(2R)-2-cyclopentyl-2-phenylpropanoyl]-1,3-oxazolidin-2-one

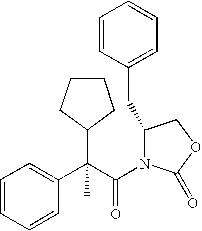

A solution of 2-cyclopentyl-2-phenyl-propionic acid (Example 1c), 4.93 g) in toluene (100 mL) was treated with thionyl chloride (30 mL) and the resultant mixture heated at 100° C. for 2 hours. The solvent was removed under reduced pressure and the residue azeotroped three times with toluene. The residue was dissolved in anhydrous tetrahydrofuran (20 mL) and added in one portion to a solution of (R)-4-benzyl-2-oxazolidinone (4.00 g) in anhydrous tetrahydrofuran (100 mL) at −78° C., which had been pre-treated at −78° C. with a 1.6 molar solution of n-butyllithium in hexanes (14.1 mL). The reaction mixture was stirred at −78° C. for 30 minutes and then at room temperature for 1 hour. At the end of this time the mixture was partitioned between ethyl acetate and saturated aqueous ammonium chloride solution, the organic layer was dried over anhydrous magnesium sulphate and evaporated under reduced pressure. Purification was by chromatography on silica gel, eluting with 10% tetrahydrofuran in iso-hexane followed by trituration of the resultant solid with iso-hexane to yield the sub-titled compound (2.97 g) as a white solid.

m/e 378 (M+H)⁺

¹H NMR (400 MHz, DMSO-D₆) δ 7.33-7.22 (m, 7H), 7.15-7.09 (m, 3H), 4.85-4.81 (m, 1H), 4.24 (t, 1H), 4.09 (q, 1H), 3.04-2.90 (m, 2H), 2.55 (t, 1H), 1.78-1.71 (m, 1H), 1.69 (s, 3H), 1.42-1.20 (m, 6H), 1.15-1.05 (m, 1H).

### b) (2R)-2-Cyclopentyl-2-phenylpropanoic acid

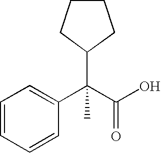

To a solution of (4R)-4-benzyl-3-[(2R)-2-cyclopentyl-2-phenylpropanoyl]-1,3-oxazolidin-2-one (Example 2a), 2.9 g) in a mixture of tetrahydrofuran (80 mL) and water (20 mL) stirred at 0° C. under nitrogen was added, dropwise a 35 wt. % solution of hydrogen peroxide in water (2.69 mL). The mixture was then treated dropwise with a solution of lithium hydroxide monohydrate (516 mg) in water (7 mL) followed by stirring for 4 hours at room temperature. At the end of this time the reaction mixture was cooled in an ice bath and treated with a solution of sodium metabisulphite (4 g) in water (30 mL). The mixture was evaporated under reduced pressure to remove the tetrahydrofuran and the residue was partitioned between diethyl ether and excess dilute aqueous sodium hydroxide. The aqueous layer was cooled and acidified by the dropwise addition of concentrated hydrochloric acid, the resultant precipitate was extracted into ethyl acetate. The ethyl acetate layer was washed with brine, dried over anhydrous magnesium sulphate and evaporated under reduced pressure. The crude product was purified by flash chromatography on a silica column, eluting with 20% ethyl acetate in iso-hexane to yield the sub-titled compound (1.7 g).

m/e 217.7 (M−H)⁻

¹H NMR (400 MHz, CDCl₃) δ 7.42-7.39 (m, 2H), 7.32 (t, 2H), 7.26-7.22 (m, 1H), 2.84-2.80 (m, 1H), 1.80-1.77 (m, 1H), 1.60-1.36 (m, 9H), 1.14-1.09 (m, 1H).

### c) 1-Methylpiperidin-4-yl (2R)-2-cyclopentyl-2-phenylpropanoate

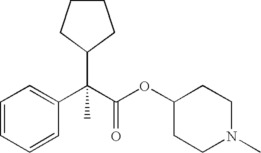

The sub-titled compound was prepared from (2R)-2-cyclopentyl-2-phenylpropionic acid (Example 2b), 500 mg) and 4-hydroxy-1-methylpiperidine (791 mg) according to the method of Example 1f). The crude product was purified by flash chromatography on a silica column, eluting with 1% triethylamine in ethyl acetate/iso-hexane (1/1) to yield the sub-titled compound (104 mg).

m/e 316 (M+H)⁺

### d) 4-{[(2R)-2-Cyclopentyl-2-phenylpropanoyl]oxy}-1,1-dimethylpiperidinium iodide

The title compound was prepared from 1-methylpiperidin-4-yl (2R)-2-cyclopentyl-2-phenylpropanoate (Example 2c), 104 mg) according to the method of Example 1g). Yield 110 mg.

m/e 330.2 (M)⁺

¹H NMR (400 MHz, DMSO-D₆) δ 7.40-7.32 (m, 4H), 7.27-7.23 (m, 1H), 4.90-4.88 (m, 1H), 3.39-3.35 (m, 2H), 3.16-3.11 (m, 2H), 3.06 (s, 6H), 2.87-2.82 (m, 1H), 2.09-2.06 (m, 2H), 1.81-1.78 (m, 2H), 1.69-1.65 (m, 1H), 1.54-1.35 (m, 9H), 1.15-1.05 (m, 1H).

### EXAMPLE 3

### 4-[(2-Cyclopropyl-2-phenylpropanoyl)oxy]-1,1-dimethylpiperidinium iodide

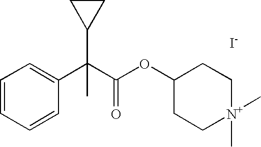

### a) 1-Methylpiperidin-4-yl 2-cyclopropyl-2-phenylpropanoate

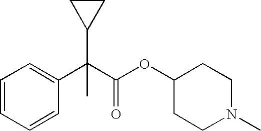

A solution of 2-cyclopropyl-2-phenyl-propionic acid (prepared according to the method outlined in Journal of Organic Chemistry (1989), 54(21), 5054-63, 110 mg) in dichloromethane (10 mL) was treated with oxalyl chloride (0.5 mL) and N,N-dimethylformamide (20 mg) and the resultant mixture stirred at room temperature for 2 hours. The solvents were removed under reduced pressure and the residue was azeotroped with dichloromethane (×3). The residue was dissolved in dichloromethane (2 mL) and added to a solution of 4-hydroxy-N-methylpiperidine (150 mg) in dichloromethane (2 mL). The reaction mixture was heated at 40° C. for 18 hours. The mixture was partitioned between dichloromethane and aqueous sodium bicarbonate solution and the organic layer was dried over anhydrous magnesium sulfate, filtered and evaporated under reduced pressure. The crude product was purified by flash chromatography on a silica column, eluting with 1% triethylamine and 4% methanol in dichloromethane to yield the sub-titled compound (67 mg).

m/e 288 (M+H)⁺

¹H NMR (400 MHz, DMSO-D₆) δ 7.37-7.31 (m, 4H), 7.26-7.22 (m, 1H), 4.75-4.73 (m, 1H), 2.35-2.23 (m, 2H), 2.22-2.15 (m, 2H), 2.10 (s, 3H), 1.80-1.70 (m, 2H), 1.52-1.43 (m, 3H), 1.17 (s, 3H), 0.59-0.56 (m, 1H), 0.48-0.45 (m, 1H), 0.35-0.28 (m, 2H).

### b) 4-[(2-Cyclopropyl-2-phenylpropanoyl)oxy]-1,1-dimethylpiperidinium iodide

The title compound was prepared from 1-methylpiperidin-4-yl 2-cyclopropyl-2-phenylpropanoate (Example 3a), 59 mg) according to the method of Example 1g). Yield 60 mg.

m/e 302.2 (M)⁺

¹H NMR (400 MHz, DMSO-D₆) δ 7.40-7.33 (m, 4H), 7.26 (t, 1H), 4.97-4.93 (m, 1H), 3.41-3.36 (m, 2H), 3.22-3.10 (m, 2H), 3.07 (s, 3H), 3.05 (s, 3H), 2.13-2.09 (m, 2H), 1.85-1.79 (m, 2H), 1.58-1.54 (m, 1H), 1.19 (s, 3H), 0.61-0.57 (m, 1H), 0.50-0.47 (m, 1H), 0.39-0.30 (m, 2H).

### EXAMPLE 4

### 4-[(2-Cyclopentyl-2-pyridin-3-ylpropanoyl)oxy]-1,1-dimethylpiperidinium iodide

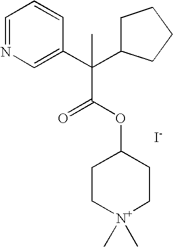

### a) Cyclopentyl(pyridin-3-yl)acetonitrile

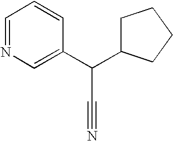

To a solution of potassium tert-butoxide (5.75 g) in tetrahydrofuran (100 mL) was added pyridin-3-yl-acetonitrile (5.5 g, 5 mL) in tetrahydrofuran (5 mL) at 0° C. After complete addition, bromocyclopentane (6.94 g) was added and the mixture stirred overnight. The mixture was poured into concentrated ammonium chloride solution and extracted into ethyl acetate (2×30 mL). The combined organic extracts were dried over anhydrous magnesium sulphate, filtered and concentrated. The residue was purified on silica gel eluting with ethyl acetate/iso-hexane (1/2 to 1/1) to give the sub-titled compound (6.7 g).

¹H NMR (300 MHz, CDCl₃) δ 8.59 (dd, 1H), 8.56 (d, 1H), 7.71 (tdt, 1H), 7.34 (dd, 1H), 3.77 (d, 1H), 2.36-2.27 (m, 1H), 1.90-1.81 (m, 1H), 1.79-1.45 (m, 6H), 1.42-1.28 (m, 1H).

### b) Methyl cyclopentyl(pyridin-3-yl)acetate

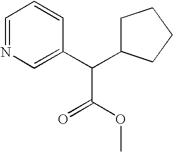

A solution of cyclopentyl-pyridin-3-yl-acetonitrile (Example 4a), 6.7 g) in concentrated hydrobromic acid (50 mL) was heated to 120° C. for 30 hours, cooled and concentrated to give a solid that was dissolved in methanol (100 mL) and concentrated sulfuric acid (1 mL) added. The reaction was stirred at reflux for 12 hours, cooled and concentrated to give the sub-titled compound (7.6 g).

¹H NMR (300 MHz, CDCl₃) δ 8.54 (d, 1H), 8.51 (dd, 1H), 7.74 (dt, 1H), 7.28-7.23 (m, 1H), 3.67 (s, 3H), 3.31 (d, 1H), 2.61-2.47 (m, 1H), 1.96-1.86 (m, 1H), 1.75-1.38 (m, 5H), 1.35-1.22 (m, 1H), 1.03-0.95 (m, 1H).

### c) Methyl 2-cyclopentyl-2-pyridin-3-ylpropanoate

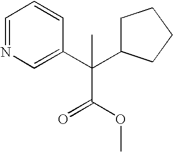

To a solution of lithium bis(trimethylsilyl)amide (1.255 g) in tetrahydrofuran (10 mL) at −70° C. was added methyl cyclopentyl(pyridin-3-yl)acetate (Example 4b, 1.096 g) in tetrahydrofuran (2 mL) and the mixture warmed to −10° C. and stirred for 1 hour before iodomethane (0.852 g, 0.382 mL) was added and the mixture stirred overnight at 0° C. The reaction was poured into water and extracted into ethyl acetate (2×30 mL). The combined organic extracts were dried over anhydrous magnesium sulphate, filtered and concentrated. The residue was purified on silica gel eluting with ethyl acetate/hexane 1/2 to give the sub-titled compound (0.8 g).

m/e 234.5 (M+H)⁺

¹H NMR (300 MHz, CDCl₃) δ 8.62 (d, 1H), 8.48 (dd, 1H), 7.67 (ddd, 1H), 7.25-7.22 (m, 1H), 3.67 (s, 3H), 2.82 (dd, 1H), 1.79-1.69 (m, 1H), 1.56 (s, 3H), 1.61-1.34 (m, 6H), 1.19-1.06 (m, 1H)

### d) 1-Methylpiperidin-4-yl 2-cyclopentyl-2-pyridin-3-ylpropanoate

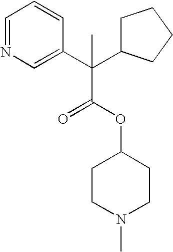

A solution of methyl 2-cyclopentyl-2-pyridin-3-ylpropanoate (Example 4c), 0.8 g) and 4-hydroxy-1-methylpiperidine (0.553 g) in heptane (40 μL) was heated with distillation until 20 mL of solvent had evaporated. The mixture was cooled and sodium (3 mg) added along with heptane (10 mL) was added and the mixture heated to reflux once more distilling away heptane and methanol. After one hour the mixture was cooled and sodium (3 mg) added along with heptane (10 mL) was added and distillation maintained for a further 1 hour. The reaction mixture was cooled and diluted with ethyl acetate and was washed with sodium hydroxide solution. The organic phase was dried with anhydrous magnesium sulphate, filtered and concentrated. The residue was purified by chromatography on silica eluting with 20% 0.7N ammonia in methanol in dichloromethane to give the titled compound (0.771 g).

m/e 317 (1+H)⁺

¹H NMR (400 MHz, CDCl₃) δ 8.65 (d, 1H), 8.47 (dd, 1H), 7.70-7.67 (m, 1H), 7.25-7.22 (m, 1H), 4.84-4.79 (m, 1H), 2.48-2.37 (m, 2H), 2.23 (s, 3H), 1.89-1.80 (m, 2H), 1.78-1.69 (m, 2H), 1.68-1.37 (m, 9H), 1.55 (s, 3H), 1.18-1.09 (m, 1H).

### e) 4-[(2-Cyclopentyl-2-pyridin-3-ylpropanoyl)oxy]-1,1-dimethylpiperidinium iodide

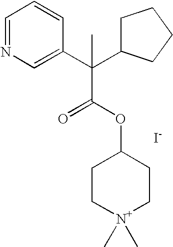

To a stirred solution of 1-methylpiperidin-4-yl 2-cyclopentyl-2-pyridin-3-ylpropanoate (Example 4d), 0.1 g) in acetonitrile (1 mL) and dichloromethane (1 mL) was added iodomethane (0.0493 g) and the reaction stirred for 2 hours. The mixture was concentrated and the residue triturated with dry diethyl ether to give the titled compound (0.135 g).

m/e 331 (M)⁺

¹H NMR (400 MHz, DMSO-D₆) δ 8.60 (s, 1H), 8.45 (d, 1H), 7.78 (d, 1H), 7.36 (t, 1H), 4.91 (s, 1H), 3.43-3.35 (m, 2H), 3.26-3.16 (m, 2H), 3.08 (s, 3H), 3.05 (s, 3H), 2.90-2.80 (m, 1H), 2.15-2.03 (m, 2H), 1.87-1.76 (m, 2H), 1.70-1.58 (m, 1H), 1.49-1.27 (m, 9H), 1.13-1.04 (m, 1H).

### EXAMPLE 5

### 4-{[2-Cyclopentyl-2-(4-hydroxyphenyl)propanoyl]oxy}-1,1-dimethylpiperidinium iodide

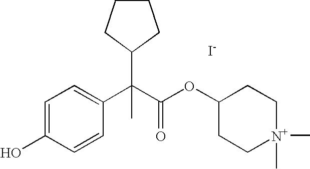

### a) Cyclopentyl(4-methoxyphenyl)acetonitrile

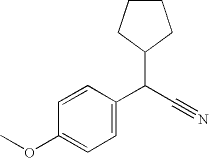

To a solution of potassium tert-butoxide (6.16 g) in tetrahydrofuran (50 mL) was dropwise added (4-methoxy-phenyl)-acetonitrile (7.35 g) in tetrahydrofuran (20 mL) over 20 minutes. The resulting solution was stirred at room temperature for 30 minutes and then bromocyclopentane (7.06 g) in tetrahydrofuran (10 mL) was added and the mixture stirred overnight. The reaction was poured into water and extracted into diethylether, dried with anhydrous magnesium sulphate, filtered and concentrated onto silica gel (30 g). The residue was added to the top of a silica column and the product isolated by a gradient elution of 4-6% ethyl acetate in iso-hexane to give the sub-titled compound (4.1 g).

¹H NMR (400 MHz, CDCl₃) δ 7.22 (d, 2H), 6.88 (d, 2H), 3.81 (s, 3H), 3.65 (d, 1H), 2.28 (q, 1H), 1.90-1.81 (m, 1H), 1.74-1.63 (m, 3H), 1.61-1.45 (m, 3H), 1.34-1.27 (m, 1H)

### b) Cyclopentyl(4-hydroxyphenyl)acetic acid

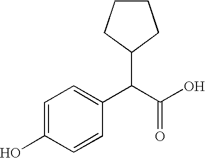

To a solution of cyclopentyl(4-methoxyphenyl)acetonitrile (Example 5a), 4.1 g) in 48% hydrobromic acid was heated to 120° C. for 2 days. The reaction mixture was cooled and the product filtered and dried to give the sub-titled compound (3.6 g).

¹H NMR (400 MHz, DMSO-D₆) δ 12.02 (s, 1H), 9.24 (s, 1H), 7.07 (d, 2H), 6.66 (d, 2H), 3.05 (d, 1H), 2.41-2.30 (m, 1H), 1.81-1.74 (m, 1H), 1.62-1.45 (m, 3H), 1.43-1.34 (m, 1H), 1.32-1.23 (m, 1H), 1.21-1.12 (m, 1H), 0.96-0.87 (m, 1H)

### c) Methyl cyclopentyl(4-hydroxyphenyl)acetate

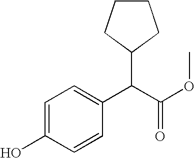

To a solution of cyclopentyl(4-hydroxyphenyl)acetic acid (Example 5b), 3.6 g) in methanol (100 mL) was added concentrated sulfuric acid (1 mL) and the reaction refluxed for 10 hours, cooled and concentrated onto silica gel. The silica was added to the top of a bed of silica and the product eluted with ethyl acetate/hexane (1/5) to give the sub-titled compound (3.64 g).

¹H NMR (400 MHz, CDCl₃) δ 7.20 (d, 2H), 6.76 (d, 2H), 4.96 (s, 1H), 3.64 (s, 3H), 3.22 (d, 1H), 2.56-2.46 (m, 1H), 1.91-1.83 (m, 1H), 1.69-1.53 (m, 3H), 1.51-1.39 (m, 2H), 1.27-1.18 (m, 1H), 1.05-0.94 (m, 1H).

### d) Methyl (4-{[tert-butyl(dimethyl)silyl]oxy}phenyl)(cyclopentyl)acetate

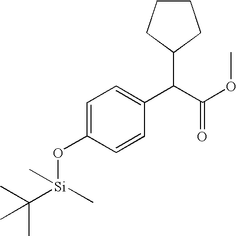

To a solution of methyl cyclopentyl(4-hydroxyphenyl)acetate (Example 5c), 3.64 g) and imidazole (1.27 g) in dichloromethane (50 mL) was added tert-butyldimethylsilyl (2.811 g) and the reaction stirred for 2 days. The reaction mixture was diluted with ether (100 mL) and was washed with 2N hydrochloric acid, the organic phase was dried with anhydrous magnesium sulphate, filtered and concentrated. The residue was purified by flash column chromatography eluting with diethyl ether/iso-hexane (1/5) to give the sub-titled compound (5.08 g) as a colourless oil.

¹H NMR (400 MHz, CDCl₃) δ 7.09 (d, 2H), 6.67 (d, 2H), 3.56 (s, 3H), 3.12 (d, 1H), 2.47-2.36 (m, 1H), 1.82-1.74 (m, 1H), 1.59-1.45 (m, 2H), 1.41-1.28 (m, 3H), 1.18-1.09 (m, 1H), 0.95-0.89 (m, 1H), 0.89 (s, 9H), 0.10 (s, 6H).

### e) Methyl 2-(4-{[tert-butyl(dimethyl)silyl]oxy}phenyl)-2-cyclopentylpropanoate

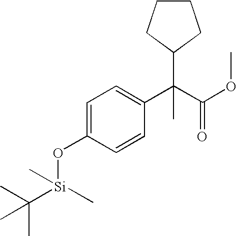

To a solution of lithium diisopropylamide (1.8M, 4.14 mL) in tetrahydrofuran (10 mL) at −70° C. was added methyl (4-{[tert-butyl(dimethyl)silyl]oxy}phenyl)(cyclopentyl)acetate (Example 5d), 1.3 g) in tetrahydrofuran (2 mL) and the mixture warmed to −10° C. and stirred for 1 hour before iodomethane (1.059 g) was added and the mixture stirred for 1 hour at 0° C. The reaction was poured into water (20 mL) and the product extracted into ethyl acetate (2×20 mL). The organic phase was dried with anhydrous magnesium sulphate, filtered and concentrated. The residue was purified by flash column chromatography eluting with 10% diethyl ether in iso-hexane to give the sub-titled compound (1.25 g).

m/e 446.6 (M+H)⁺

¹H NMR (400 MHz, CDCl₃) δ 7.19 (d, 2H), 6.75 (d, 2H), 3.63 (s, 3H), 2.77 (t, 1H), 1.76-1.65 (m, 1H), 1.59-1.02 (m, 7H), 1.48 (s, 3H), 0.97 (s, 9H), 0.19 (s, 6H).

### f) 1-Methylpiperidin-4-yl 2-(4-{[tert-butyl(dimethyl)silyl]oxy}phenyl)-2-cyclopentylpropanoate

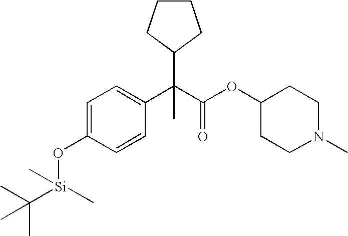

A solution of methyl 2-(4-{[tert-butyl(dimethyl)silyl]oxy}phenyl)-2-cyclopentylpropanoate (example 5e), 1.25 g) and 4-hydroxy-1-methylpiperidine (0.596 g) in heptane (40 mL) was heated with distillation until 20 mL of solvent had evaporated. The mixture was cooled and sodium (3 mg) added along with heptane (10 mL) was added and the mixture heated to reflux distilling away heptane and methanol. After one hour the mixture was cooled and sodium (3 mg) added along with heptane (10 mL) was added and distillation maintained for a further one hour. The reaction mixture was cooled and diluted with ethyl acetate and washed with sodium hydroxide solution. The organic phase was dried with anhydrous magnesium sulphate, filtered and concentrated. The residue was purified by chromatography on silica eluting with 10-20% 0.7N ammonia in methanol in ethyl acetate to give the sub-titled compound (0.713 g).

m/e 446.6 (M+H)⁺

¹H NMR (400 MHz, CDCl₃) δ 7.22 (d, 2H), 6.75 (d, 2H), 4.79 (s, 1H), 2.83 (quintet, 1H), 2.43-2.33 (m, 2H), 2.28-2.19 (m, 2H), 2.22 (s, 3H), 1.85-1.67 (m, 3H), 1.67-1.32 (m, 8H), 1.47 (s, 3H), 1.16-1.06 (m, 1H), 0.97 (s, 9H), 0.18 (s, 6H).

### g) 1-Methylpiperidin-4-yl 2-cyclopentyl-2-(4-hydroxyphenyl)propanoate

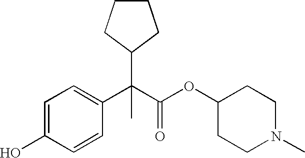

To a solution of 1-methylpiperidin-4-yl 2-(4-{[tert-butyl(dimethyl)silyl]oxy}phenyl)-2-cyclopentylpropanoate (Example 5H), 0.71 g) in methanol (3 mL) was added 2 N hydrochloric acid (10 mL) and the reaction stirred at room temperature for 4 hours. The mixture was concentrated and the residue was dissolved in methanol and passed down a SCX column eluting with methanol. The product was recovered by elution with 3N ammonia in methanol to give the titled compound (0.52 g) as a white solid after trituration with 20% ether in hexane.

m.p. 128-130° C.

m/e 332 (M+H)⁺

¹H NMR (400 MHz, DMSO-D₆) δ 9.27 (s, 1H), 7.12 (d, 2H), 6.69 (d, 2H), 4.72-4.64 (m, 1H), 2.40-2.29 (m, 2H), 2.25-2.15 (m, 2H), 2.12 (s, 3H), 1.77-1.67 (m, 2H), 1.66-1.58 (m, 1H), 1.54-1.26 (m, 11H), 1.11-1.00 (m, 1H).

### h) 4-{[2-Cyclopentyl-2-(4-hydroxyphenyl)propanoyl]oxy}-1,1-dimethylpiperidinium iodide

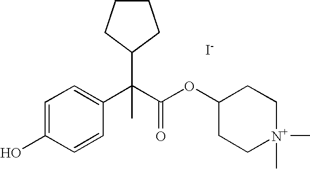

To a solution of 1-methylpiperidin-4-yl 2-cyclopentyl-2-(4-hydroxyphenyl)propanoate (Example 5g), 0.1 g) in acetonitrile (2 mL) was added iodomethane (0.2 mL) and the resulting solution stirred overnight, concentrated and triturated with diethyl ether to give the titled compound (0.124 g).

m/e 346 (M)⁺

¹H NMR (400 MHz, DMSO-D₆) δ 9.33 (s, 1H), 7.18 (d, 2H), 6.71 (d, 2H), 4.89-4.81 (m, 1H), 3.43-3.33 (m, 2H), 3.20-3.09 (m, 2H), 3.07 (s, 3H), 3.06 (s, 3H), 2.82 (m, 1H), 2.13-2.01 (m, 2H), 1.84-1.74 (m, 2H), 1.70-1.60 (m, 1H), 1.56-1.28 (m, 9H), 1.11-1.05 (m, 1H).

### EXAMPLE 6

### 4-[(2-Cyclobutyl-2-phenylpropanoyl)oxy]-1,1-dimethylpiperidinium iodide

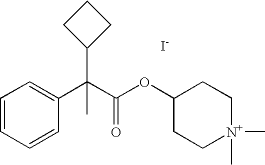

### a) (E/Z)-2-Cyclobutyl-2-phenylvinyl methyl ether

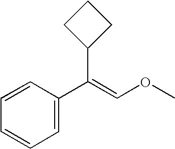

To a solution of (methoxymethyl)triphenylphosphonium chloride (6.8 g) in tetrahydrofuran (50 mL) at 0° C. was added n-butyllithium over 5 minutes. The mixture was stirred at 0° C. for 10 minutes and cyclobutyl phenyl ketone (3.2 g) added. The mixture was allowed to warm to room temperature overnight, then poured into water and extracted with diethyl ether (3×20 mL). The combined organic extracts were dried with anhydrous magnesium sulphate, filtered and concentrated. The resulting oil was purified by chromatography on silica eluting with 3% ethyl acetate in isohexane to give the sub-titled compound (1.6 g) as a mixture of E/Z isomers (˜1:1).

¹H NMR (400 MHz, CDCl₃) [symbol] 7.43-7.11 (m, 5H), 6.02-5.99 (m, 0.5H), 5.99-5.96 (m, 0.5H), 3.64 (s, 1.5H), 3.63 (s, 1.5H), 3.61-3.51 (m, 0.5H), 3.35-3.25 (m, 0.5H), 2.20-1.60 (m, 6H).

### b) Cyclobutyl(phenyl)acetic acid

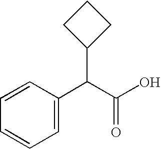

To a solution of (E/Z)-2-cyclobutyl-2-phenylvinyl methyl ether (Example 6a) (1.3 g) in tetrahydrofuran (10 mL) was added formic acid (3 mL). The mixture was warmed to 60° C. for 4 hours and then allowed to cool to room temperature. The mixture was poured into saturated sodium bicarbonate solution (20 mL) and extracted with ethyl acetate (3×20 mL). The organic phase was dried with anhydrous magnesium sulphate, filtered and concentrated to yield a crude oil which was re-dissolved in a mixture of dichloromethane (23 mL) and water (6 mL) and 1-methyl-1-cyclohexene (4.36 g) added. The solution was cooled to 5° C. and aqueous sulphamic acid solution (1M, 8.1 mL) added dropwise. To the rapidly stirred mixture at 5° C. was added aqueous sodium chlorite solution (1M, 27 mL) over 30 minutes. The mixture was allowed to warm to room temperature and poured into saturated sodium bicarbonate solution (20 mL) and extracted with dichloromethane (2×20 mL). The aqueous phase was adjusted to pH 1 with dilute hydrochloric acid and extracted with diethyl ether (3×25 mL). The combined ethereal extract was dried with anhydrous magnesium sulphate, filtered and concentrated. The crude product was recrystallised from iso-hexane to afford the sub-titled product as a white solid (0.65 g).

¹H NMR (400 MHz, CDCl₃) [symbol] 7.34-7.21 (m, 5H), 3.55 (d, 1H), 3.04-2.90 (m, 1H), 2.27-2.17 (m, 1H), 1.89-1.78 (m, 4H), 1.65-1.54 (m, 1H).

### c) Methyl cyclobutyl(phenyl)acetate

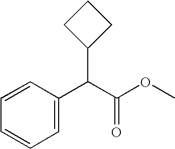

To a solution of cyclobutyl(phenyl)acetic acid (Example 6b) (650 mg) and pyridine (0.7 mL) in toluene (5 mL) at room temperature was added thionyl chloride (1 mL) dropwise over 5 minutes. The mixture was stirred for 2 hours and then methanol (2 mL) added. The mixture was stirred for a further 30 minutes and then poured into diethyl ether (10 mL) and aqueous hydrochloric acid solution (1M, 10 mL). The mixture was shaken and the organic layer separated. The aqueous layer was extracted with diethyl ether (2×20 mL) and the combined organic extracts dried with anhydrous magnesium sulphate, filtered and concentrated to afford the sub-titled compound as an oil (650 mg).

¹H NMR (400 MHz, CDCl₃) [symbol] 7.34-7.21 (m, 5H), 3.64 (s, 3H), 3.54 (d, 1H), 3.04-2.92 (m, 1H), 2.25-2.13 (m, 1H), 1.92-1.75 (m, 4H), 1.61-1.54 (m, 1H).

### d) Methyl 2-cyclobutyl-2-phenylpropanoate

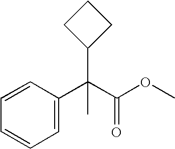

To a solution of dry diisopropylamine (490 mg) in tetrahydrofuran (5 mL) at −78° C. was added n-butyllithium (2.5M, 2 mL). The mixture was allowed to warm to −30° C. for 15 minutes and then re-cooled to −78° C. whereupon methyl cyclobutyl(phenyl)acetate (Example 6c, 650 mg) in tetrahydrofuran (2 mL) was added. The mixture was warmed to −30° C. and stirred for 30 minutes before iodomethane (670 mg) was added and the mixture allowed to warm to 10° C. over 2 hours. The reaction was poured into aqueous ammonium chloride solution and extracted into diethyl ether (2×30 TL). The combined organic extracts were dried over anhydrous magnesium sulphate, filtered and concentrated to give the sub-titled compound (585 mg) as an oil.

¹H NMR (400 MHz, CDCl₃) [symbol] 7.35-7.18 (m, 5H), 3.66 (s, 3H), 3.20-3.06 (m, 1H), 2.00-1.74 (m, 5H), 1.70-1.62 (m, 1H), 1.53 (s, 3H).

### e) 1-Methylpiperidin-4-yl 2-cyclobutyl-2-phenylpropanoate

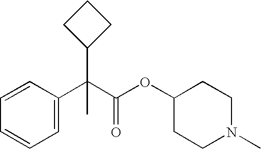

The sub-titled compound was prepared from methyl 2-cyclobutyl-2-phenylpropanoate (Example 6d) (350 mg) and 4-hydroxy-1-methylpiperidine (480 mg) according to the method of Example 4d. The crude product was purified by flash chromatography on a silica column, eluting with 1% triethylamine in ethyl acetate/iso-hexane (1/1) to yield the sub-titled compound (280 mg).

m/e 302.2 (M+H)⁺

¹H NMR (400 MHz, CDCl₃) [symbol] 7.32-7.17 (m, 5H), 4.87-4.78 (m, 1H), 3.22-3.10 (m, 1H), 2.49-2.35 (m, 2H), 2.29-2.16 (m, 5H), 2.00-1.91 (m, 2H), 1.90-1.77 (m, 4H), 1.71-1.58 (m, 4H), 1.52 (s, 3H).

### f) 4-[(2-Cyclobutyl-2-phenylpropanoyl)oxy]-1,1-dimethylpiperidinium iodide

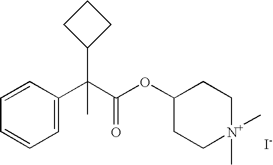

To a solution of 1-methylpiperidin-4-yl 2-cyclobutyl-2-phenylpropanoate (Example 6e) (50 mg) in acetonitrile (3 mL) was added iodomethane (0.2 mL) and the resulting solution stirred overnight, concentrated and triturated with diethyl ether and isohexane to give the titled compound (50 mg).

m.p. 133-134° C.

m/e 316.2 (M⁺)

¹H NMR (400 MHz, DMSO-D₆) δ 7.37-7.30 (m, 2H), 7.28-7.21 (m, 3H), 4.96-4.89 (m, 1H), 3.45-3.35 (m, 2H), 3.23-3.11 (m, 3H), 3.07 (s, 3H), 3.06 (s, 3H), 2.15-2.04 (m, 2H), 1.95-1.74 (m, 7H), 1.67-1.57 (m, 1H), 1.49 (s, 3H).

### EXAMPLE 7

### 4-{[2-Cyclopentyl-2-(4-methoxyphenyl)propanoyl]oxy}-1,1-dimethylpiperidinium iodide

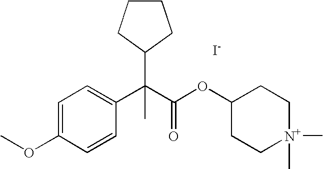

To a solution of 1-methylpiperidin-4-yl 2-cyclopentyl-2-(4-hydroxyphenyl)propanoate (Example 5g) (107 mg) in tetrahydrofuran (3 mL) was added sodium hydride (60% in oil, 13 mg). The mixture was stirred for 5 minutes at room temperature and iodomethane (245 mg) added. The mixture was stirred at room temperature for 18 hours and then evaporated under reduced pressure. The crude product was purified on a C18 column, eluting with water/methanol (99/1→50/50) to afford the titled product (38 mg).

m/e 360.2 (M⁺)

¹H NMR (400 MHz, DMSO-D₆) δ 7.30 (dd, 2H), 6.89 (dd, 2H), 4.93-4.81 (m, 1H), 3.74 (s, 3H), 3.43-3.34 (m, 2H), 3.21-3.11 (m, 2H), 3.07 (d, 6H), 2.84 (quintet, 1H), 2.14-2.00 (m, 2H), 1.86-1.73 (m, 2H), 1.73-1.60 (m, 1H), 1.57-1.48 (m, 4H), 1.45 (s, 3H), 1.41-1.28 (m, 2H), 1.14-1.02 (m, 1H).

### EXAMPLE 8

### 4-{[2-Cyclopentyl-2-(2-thienyl)propanoyl]oxy}-1,1-dimethylpiperidinium iodide

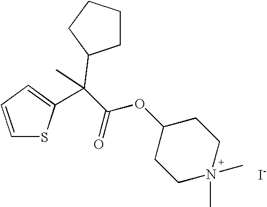

### a) Ethyl cyclopentyl(2-thienyl)acetate

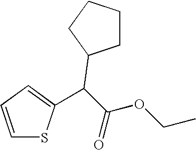

A solution of n-butyllithium in hexanes (1.7M, 62 mL) was added over 35 minutes to a solution of N,N-diisopropylamine (15 mL) in tetrahydrofuran (120 mL) at −78° C. The solution was stirred for 30 minutes and then treated with a solution of ethyl thiophene-2-acetate (13.2 mL) in tetrahydrofuran (120 mL), added over 30 minutes. The resulting dark orange solution was stirred at −78° C. for 30 minutes and then treated with a solution of bromocyclopentane (14.2 mL) in tetrahydrofuran (60 mL), added over 20 minutes, to give a brown suspension. The cooling bath was removed and the mixture was stirred overnight, allowing it to warm to room temperature. The mixture was then stirred at 40° C. for 1.5 hours, at 50° C. for 7.5 hours and was finally stirred at room temperature overnight. The mixture was quenched by the careful addition of 2 molar aqueous hydrochloric acid (10 mL) with cooling in ice-water. The mixture was then poured into 2 molar hydrochloric acid and extracted twice with diethyl ether. The combined organic extracts were washed twice with water, once with brine, then dried (MgSO₄) and concentrated onto silica (100 mL) in vacuo. The resulting powder was loaded onto a column of silica (1500 μL) and eluted with a 2% solution of diethyl ether in isohexane to afford the subtitled compound as a pale yellow oil (5.30 g).

m/e 238 (M)⁺, 97 (100%).

¹H NMR (400 MHz, CDCl₃) δ 7.18-7.21 (m, 1H), 6.91-6.96 (m, 2H), 4.08-4.23 (m, 2H), 3.59 (d, 1H), 2.44-2.56 (m, 1H), 1.80-1.89 (m, 1H), 1.45-1.72 (m, 5H), 1.22-1.34 (m, 1H), 1.25 (t, 3H), 1.07-1.19 (m, 1H).

### b) Ethyl cyclopentyl[5-(trimethylsilyl)-2-thienyl]acetate

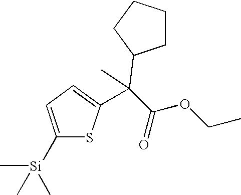

A solution of 2,2,6,6-tetramethylpiperidine (4.5 mL) in tetrahydrofuran (50 mL) was cooled to −78° C. and treated with a solution of n-butyllithium in hexanes (1.7 M, 15.7 mL), added over 20 minutes. The solution was stirred at −78° C. for 15 minutes and then at 0° C. for 15 minutes. After cooling back to −78° C. the solution was treated with a solution of ethyl cyclopentyl(2-thienyl)acetate (Example 8a) (5.29 g) in tetrahydrofuran (15 mL), added over 30 minutes. The resulting orange solution was stirred at −78° C. for 1 hour and was then treated with a solution of chlorotrimethylsilane (3.4 mL) in tetrahydrofuran (15 mL), added over 15 minutes. The resulting dark brown solution was stirred overnight, allowing it to slowly warm to room temperature. The solution was then poured into 2 molar aqueous hydrochloric acid and extracted twice with diethyl ether. The combined organic extracts were washed with brine, dried (MgSO₄) and concentrated onto silica (50 mL) in vacuo. The resulting powder was loaded onto a column of silica (1000 mL) and eluted with a 1% solution of diethyl ether in isohexane to afford the subtitled compound as a pale yellow oil (3.15 g).

m/e 310 (M)⁺, 237 (100%).

¹H NMR (400 MHz, CDCl₃) δ 7.06 (d, 1H), 7.00 (d, 1H), 4.07-4.23 (m, 2H), 3.62 (d, 1H), 2.44-2.56 (m, 1H), 1.80-1.89 (m, 1H), 1.44-1.72 (m, 5H), 1.20-1.33 (m, 1H), 1.25 (t, 3H), 1.07-1.19 (m, 1H), 0.29 (s, 9H).

### c) Ethyl 2-cyclopentyl-2-[5-(trimethylsilyl)-2-thienyl]propanoate

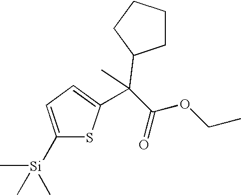

A solution of n-butyllithium in hexanes (1.7M, 11.5 mL) was added over 25 minutes to a solution of N,N-diisopropylamine (2.76 mL) in tetrahydrofuran (35 mL) at −78° C. The solution was stirred at −78° C. for 20 minutes and then treated with a solution of ethyl cyclopentyl[5-(trimethylsilyl)-2-thienyl]acetate (Example 8b) (5.05 g) in anhydrous tetrahydrofuran (20 mL), added over 30 minutes. The resulting yellow solution was stirred at −78° C. for 30 minutes and then treated with iodomethane (1.2 mL), added in one portion. The cooling bath was removed and the yellow solution was slowly allowed to warm to room temperature with stirring over 75 minutes. The solution was then poured into 2 molar aqueous hydrochloric acid and extracted twice with diethyl ether. The combined organic extracts were washed twice with 2 molar aqueous hydrochloric acid, twice with 10% aqueous sodium metabisulphite solution, once with brine, then dried (MgSO₄) and concentrated onto silica (50 mL) in vacuo. The resulting powder was loaded onto a column of silica (1500 mL) and eluted with a 1% solution of diethyl ether in isohexane to afford the subtitled compound as a colourless oil (3.90 g).

m/e 324 (M)⁺, 255 (100%).

¹H NMR (400 MHz, CDCl₃) δ 7.06 (d, 1H), 7.01 (d, 1H), 4.10-4.18 (m, 2H), 2.78-2.88 (m, 1H), 1.63-1.73 (m, 1H), 1.46-1.63 (m, 8H), 1.30-1.42 (m, 1H), 1.19-1.30 (m, 1H), 1.24 (t, 3H), 0.29 (s, 9H).

### d) Ethyl 2-cyclopentyl-2-(2-thienyl)propanoate

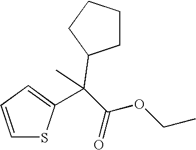

A solution of ethyl 2-cyclopentyl-2-[5-(trimethylsilyl)-2-thienyl]propanoate (Example 8c) (3.89 g) in anhydrous tetrahydrofuran (100 mL) was treated with a solution of tetrabutylammonium fluoride in tetrahydrofuran (1M, 24 mL), added over 5 minutes, and the resulting solution was stirred at room temperature for 6 hours. The brown solution was then poured into water and extracted twice with diethyl ether. The combined organic extracts were washed twice with water, once with brine, then dried (MgSO₄) and concentrated onto silica (50 mL) in-vacuo. The resulting powder was loaded onto a column of silica (1500 mL) and eluted with a 2% solution of diethyl ether in isohexane to afford the subtitled compound as a colourless oil (2.78 g).

m/e 252 (M)⁺, 179 (100%).

¹H NMR (400 MHz, CDCl₃) δ 7.17-7.20 (m, 1H), 6.95-6.98 (m, 1H), 6.91-6.94 (m, 1H), 4.10-4.18 (m, 2H), 2.78-2.88 (m, 1H), 1.64-1.73 (m, 1H), 1.46-1.63 (m, 8H), 1.30-1.41 (m, 1H), 1.19-1.29 (m, 1H), 1.24 (t, 3H).

### e) 1-Methylpiperidin-4-yl 2-cyclopentyl-2-(2-thienyl)propanoate

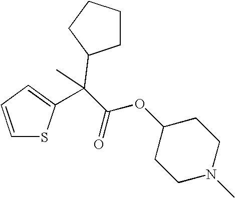

Prepared by the method of Example 4d using ethyl 2-cyclopentyl-2-(2-thienyl)propanoate (Example 8d) (0.106 g) in place of methyl 2-cyclopentyl-2-pyridin-3-ylpropanoate.

Purification was by flash chromatography on silica eluted with triethylamine:ethanol:isohexane (1:5:94). Yield 0.076 g.

m/e 322 (M+H)⁺, 100%.

¹H NMR (400 MHz, CDCl₃) δ 7.17-7.19 (m, 1H), 6.96-6.98 (m, 1H), 6.91-6.94 (m, 1H), 4.77-4.85 (m, 1H), 2.80-2.90 (m, 1H), 2.41-2.55 (bm, 2H), 2.22-2.35 (bm, 2H), 2.26 (s, 3H), 1.80-1.93 (m, 2H), 1.62-1.75 (m, 4H), 1.47-1.62 (m, 4H), 1.56 (s, 3H), 1.31-1.44 (m, 1H), 1.19-1.29 (m, 1H).

### f) 4-{[2-Cyclopentyl-2-(2-thienyl)propanoyl]oxy}-1,1-dimethylpiperidinium iodide

Prepared by the method of Example 1g using 1-methylpiperidin-4-yl 2-cyclopentyl-2-(2-thienyl)propanoate (Example 8e) (0.075 g) to yield 0.096 g.

m/e 336 (M−I⁻)⁺, 100%.

¹H NMR (400 MHz, DMSO-D₆) δ 7.44-7.47 (m, 1H), 7.06-7.09 (m, 1H), 6.97-7.01 (m, 1H), 4.85-4.92 (m, 1H), 3.35-3.46 (m, 2H), 3.14-3.30 (m, 2H), 3.12 (s, 3H), 3.07 (s, 3H), 2.82-2.92 (m, 1H), 2.05-2.19 (m, 2H), 1.78-1.92 (bm, 2H), 1.60-1.70 (m, 1H), 1.43-1.59 (m, 5H), 1.52 (s, 3H), 1.26-1.37 (m, 1H), 1.14-1.26 (m, 1H).

### EXAMPLE 9

### 4-{[2-Cyclopentyl-2-(5-methyl-2-thienyl)propanoyl]oxy}-1,1-dimethylpiperidinium iodide

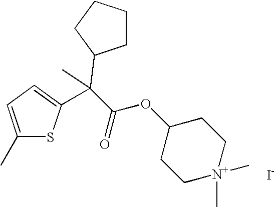

### a) Ethyl 2-cyclopentyl-2-(5-methyl-2-thienyl)propanoate

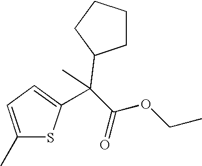

A solution of n-butyllithium in hexanes (1.8M, 4.62 mL) was added over 10 minutes to a solution of N,N-diisopropylamine (1.17 mL) in tetrahydrofuran (10 mL) at −78° C. The solution was stirred for 15 minutes and then treated with a solution of ethyl cyclopentyl(2-thienyl)acetate (Example 8a) (1.65 g) in tetrahydrofuran (10 mL), added over 10 minutes. The yellow solution was stirred at −78° C. for 30 minutes and then treated with iodomethane (2.11 mL), added in one portion. The cooling bath was removed and the orange solution was slowly allowed to warm to room temperature with stirring over 90 minutes. The resulting brown mixture was then poured into 2 molar aqueous hydrochloric acid and extracted twice with diethyl ether. The combined organic extracts were washed with brine, dried (MgSO₄) and concentrated to afford a brown oil. (1.82 g).

A solution of n-butyllithium in hexanes (1.8M, 4.62 mL) was added over 5 minutes to a solution of N,N-diisopropylamine (1.17 mL) in tetrahydrofuran (10 mL) at −78° C. The solution was stirred for 15 minutes and then treated with a solution of the brown oil prepared above (1.82 g) in tetrahydrofuran (10 mL), added over 10 minutes. The brown solution was stirred at −78° C. for 30 minutes and then treated with iodomethane (2.11 mL), added in one portion. The cooling bath was removed and the mixture was slowly allowed to warm to room temperature with stirring over 3 hours. The brown mixture was quenched by the dropwise addition of 2 molar hydrochloric acid (5 mL) and was then poured into 2 molar hydrochloric acid and extracted twice with diethyl ether. The combined organic extracts were washed twice with 10% aqueous sodium metabisulphite, twice with water and once with brine, then dried (MgSO₄) and concentrated onto silica (20 mL) in-vacuo. The resulting powder was purified by flash chromatography on silica eluted with a 3% solution of diethyl ether in isohexane to afford the subtitled compound as a colourless oil (1.35 g).

m/e 266 (M)⁺, 197 (100%).

¹H NMR (300 MHz, CDCl₃) δ 6.72 (d, 1H), 6.54-6.58 (m, 1H), 6.91-6.94 (m, 1H), 4.08-4.19 (m, 2H), 2.72-2.87 (m, 1H), 2.43 (d, 3H), 1.43-1.74 (m, 8H), 1.16-1.42 (m, 2H), 1.24 (t, 3H).

### b) 1-Methylpiperidin-4-yl 2-cyclopentyl-2-(5-methyl-2-thienyl)propanoate

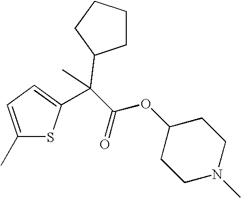

Prepared by the method of Example 4d using ethyl 2-cyclopentyl-2-(5-methyl-2-thienyl)propanoate (Example 9a) (0.536 g). Purification was by preparative HPLC using a Phenomenex Gemini 5 um C18 110A AXIA column eluted with 0.1% aqueous ammonia solution and acetonitrile. Yield 0.396 g.

m/e 336 (M+H)⁺, 100%.

¹H NMR (400 MHz, CDCl₃) δ 6.72 (d, 1H), 6.54-6.57 (m, 1H), 4.76-4.84 (m, 1H), 2.75-2.85 (m, 1H), 2.44-2.57 (bm, 2H), 2.42 (s, 3H), 2.22-2.34 (bm, 2H), 2.26 (s, 3H), 1.81-1.92 (m, 2H), 1.62-1.76 (m, 4H), 1.46-1.62 (m, 4H), 1.51 (s, 3H), 1.29-1.40 (m, 1H), 1.18-1.29 (m, 1H).

### c) 4-{[2-Cyclopentyl-2-(5-methyl-2-thienyl)propanoyl]oxy}-1,1-dimethylpiperidinium iodide

Prepared by the method of Example 1g using 1-methylpiperidin-4-yl 2-cyclopentyl-2-(5-methyl-2-thienyl)propanoate (Example 9b) (0.098 g) to yield 0.133 g.

m/e 350 (M⁺), 100%.

¹H NMR (400 MHz, DMSO-D₆) δ 6.81-6.85 (m, 1H), 6.63-6.67 (m, 1H), 4.83-4.91 (m, 1H), 3.36-3.47 (m, 1H), 3.15-3.35 (m, 2H), 3.12 (s, 3H), 3.08 (s, 3H), 2.75-2.87 (m, 2H), 2.39 (s, 3H), 2.04-2.19 (m, 2H), 1.78-1.92 (bm, 2H), 1.58-1.68 (m, 1H), 1.40-1.58 (m, 5H), 1.46 (s, 3H), 1.13-1.35 (m, 2H).

### EXAMPLE 10

### 4-{[2-(3-Bromophenyl)-2-cyclopentylpropanoyl]oxy}-1,1-dimethylpiperidinium iodide

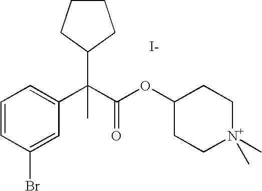

### a) (3-Bromophenyl)cyclopentylacetonitrile

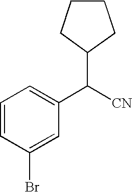

The sub-titled compound was prepared from 3-bromophenylacetonitrile (11.76 g) and cyclopentyl bromide (9.84 g) according to the method of Example 4a. The crude product was purified by flash chromatography on a silica column, eluting with 25% dichloromethane in iso-hexane to yield the sub-titled compound (10.5 g).

¹H NMR (399.824 MHz, CDCl₃) δ 7.49-7.43 (m, 2H), 7.29-7.22 (m, 2H), 3.69 (d, 1H), 2.35-2.23 (m, 1H), 1.91-1.81 (m, 1H), 1.78-1.44 (m, 6H), 1.40-1.29 (m, 1H).

### b) Methyl (3-bromophenyl)cyclopentylacetate

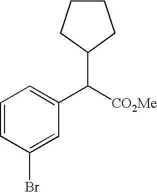

The sub-titled compound was prepared from (3-bromophenyl)cyclopentylacetonitrile (Example 10a) (10.5 g) according to the method of Example 4b). The crude product was isolated as a pale brown oil (11.1 g).

¹H NMR (399.824 MHz, CDCl₃) δ 7.50 (s, 1H), 7.38 (d, 1H), 7.27 (d, 1H), 7.17 (t, 1H), 3.66 (s, 3H), 3.24 (d, 1H), 2.58-2.45 (m, 1H), 1.94-1.84 (m, 1H), 1.72-1.39 (m, 5H), 1.29-1.19 (m, 1H), 1.04-0.93 (m, 1H).

### c) Methyl 2-(3-bromophenyl)-2-cyclopentylpropanoate

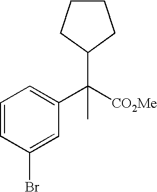

The sub-titled compound was prepared from methyl (3-bromophenyl)(cyclopentyl)acetate (Example 10b) (5.94 g) and iodomethane (1.99 mL) according to the method of Example 4c. On completion of the reaction, the reaction mixture was partitioned between saturated ammonium chloride solution and diethyl ether. The combined organic extracts were washed with water and brine, then dried over anhydrous magnesium sulphate, filtered and concentrated. The crude product was purified by flash chromatography on a silica column, eluting with 25% dichloromethane in iso-hexane to yield the sub-titled compound (5.54 g).

¹H NMR (399.824 MHz, CDCl₃) δ 7.49 (s, 1H), 7.36 (d, 1H), 7.27 (d, 1H), 7.17 (t, 1H), 3.65 (s, 3H), 2.81-2.71 (m, 1H), 1.79-1.69 (m, 1H), 1.61-1.46 (m, 3H), 1.50 (s, 3H), 1.49-1.31 (m, 3H), 1.48-1.31 (m, 1H).

### d) 1-Methylpiperidin-4-yl 2-(3-bromophenyl)-2-cyclopentylpropanoate

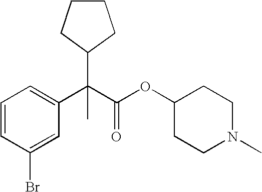

The sub-titled compound was prepared from methyl 2-(3-bromophenyl)-2-cyclopentylpropanoate (Example 10c) (1.26 g) and 4-hydroxy-1-methylpiperidine (0.94 g) according to the method of Example 4d. The crude product (1.47 g) was used without further purification.

¹H NMR (299.946 MHz, CDCl₃) δ 7.55-7.51 (m, 1H), 7.37-7.32 (m, 1H), 7.32-7.27 (m, 1H), 7.16 (t, 1H), 4.86-4.76 (m, 1H), 2.86-2.72 (m, 1H), 2.47-2.33 (m, 2H), 2.30-2.16 (m, 2H), 2.23 (s, 3H), 1.89-1.76 (m, 2H), 1.77-1.21 (m, 8H), 1.49 (s, 3H), 1.18-1.03 (m, 2H).

### e) 4-{[2-(3-Bromophenyl)-2-cyclopentylpropanoyl]oxy}-1,1-dimethylpiperidinium iodide

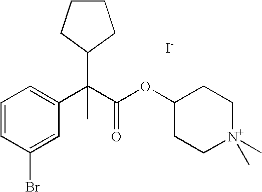

To a stirred solution of 1-methylpiperidin-4-yl 2-(3-bromophenyl)-2-cyclopentylpropanoate (Example 10d) (0.15 g) in acetonitrile (3 mL) was added iodomethane (0.047 mL) and the reaction stirred for 2 hours. The mixture was concentrated and the residue triturated with dry diethyl ether to give the title compound (0.19 g).

m/e 410/412 (M)⁺

¹H NMR (399.826 MHz, DMSO) δ 7.55 (s, 1H), 7.47 (d, 1H), 7.40 (d, 1H), 7.32 (t, 1H), 4.92 (s, 1H), 3.45-3.17 (m, 4H), 3.10 (s, 3H), 3.07 (s, 3H), 2.87-2.77 (m, 1H), 2.16-2.04 (m, 2H), 1.87-1.76 (m, 2H), 1.72-1.61 (m, 1H), 1.57-1.21 (m, 9H), 1.16-1.04 (m, 1H).

### EXAMPLE 11

### 4-{[2-(4-Bromophenyl)-2-cyclopentylpropanoyl]oxy}-1,1-dimethylpiperidinium iodide

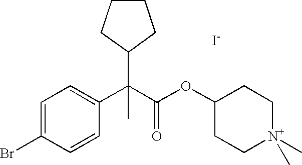

### a) Methyl (4-bromophenyl)cyclopentylacetate

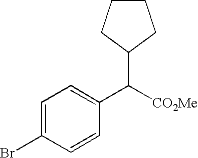

The preparation was performed by treatment of 4-bromophenylacetonitrile (10.0 g) with cyclopentyl bromide (10.64 g) according to the method of Example 4a. The crude product (12 g) was used without further purification and was subsequently converted to the corresponding ester using the procedure outlined in Example 4b. The crude product was isolated as a pale brown oil (6.4 g).

¹H NMR (399.824 MHz, CDCl₃) δ 7.43 (d, 2H), 7.22 (d, 2H), 3.65 (s, 3H), 3.24 (d, 1H), 2.57-2.44 (m, 1H), 1.94-1.84 (m, 1H), 1.71-1.37 (m, 5H), 1.30-1.18 (m, 1H), 1.03-0.92 (m, 1H).

### b) Methyl 2-(4-bromophenyl)-2-cyclopentylpropanoate

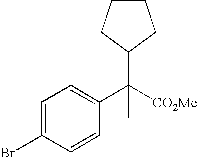

The sub-titled compound was prepared from methyl (4-bromophenyl)(cyclopentyl)acetate (Example 1a) (6.4 g) and iodomethane (2.15 mL) according to the method of Example 4c. On completion of the reaction, the reaction mixture was partitioned between saturated ammonium chloride solution and diethyl ether. The combined organic extracts were washed with water and brine then dried over anhydrous magnesium sulphate, filtered and concentrated. The crude product was purified by flash chromatography on a silica column, eluting with 33%-50% dichloromethane in iso-hexane to yield the sub-titled compound (6.3 g).

¹H NMR (399.824 MHz, CDCl₃) δ 7.42 (d, 2H), 7.22 (d, 2H), 3.64 (s, 3H), 2.81-2.71 (m, 1H), 1.77-1.68 (m, 1H), 1.61-1.45 (m, 311), 1.49 (s, 3H), 1.46-1.30 (m, 3H), 1.14-1.03 (m, 1H).

### c) 1-Methylpiperidin-4-yl 2-(4-bromophenyl)-2-cyclopentylpropanoate

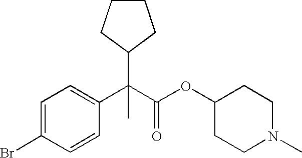

The sub-titled compound was prepared from methyl 2-(4-bromophenyl)-2-cyclopentylpropanoate (Example 11b) (0.311 g) and 4-hydroxy-1-methylpiperidine (0.23 g) according to the method of Example 4d. The crude product was purified by flash chromatography on a silica column, eluting with 1% triethylamine in ethyl acetate to yield the sub-titled compound (0.27 g).

¹H NMR (399.824 MHz, CDCl₃) δ 7.41 (d, 2H), 7.24 (d, 2H), 4.82-4.75 (m, 1H), 2.84-2.74 (m, 1H), 2.49-2.37 (m, 2H), 2.28-2.21 (m, 2H), 2.23 (s, 3H), 1.87-1.77 (m, 2H), 1.77-1.68 (m, 2H), 1.68-1.31 (m, 7H), 1.48 (s, 3H), 1.16-1.04 (m, 1H).

### d) 4-{[2-(4-bromophenyl)-2-cyclopentylpropanoyl]oxy}-1,1-dimethylpiperidinium iodide

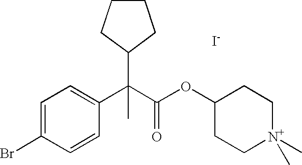

To a stirred solution of 1-methylpiperidin-4-yl 2-(4-bromophenyl)-2-cyclopentylpropanoate (Example 11c) (0.2 g) in acetonitrile (4 mL) was added iodomethane (0.095 mL) and the reaction stirred for 2 hours. The mixture was concentrated and the residue triturated with dry diethyl ether to give the title compound (0.05 g).

m/e 410/412 (M)⁺

¹H NMR (399.826 MHz, DMSO) δ 7.53 (d, 2H), 7.34 (d, 2H), 4.94-4.86 (m, 1H), 3.45-3.35 (m, 2H), 3.26-3.15 (m, 2H), 3.08 (s, 3H), 3.07 (s, 3H), 2.85-2.75 (m, 1H), 2.14-2.03 (m, 2H), 1.88-1.75 (m, 2H), 1.72-1.61 (m, 1H), 1.59-1.42 (m, 4H), 1.47 (s, 3H), 1.42-1.31 (m, 2H), 1.15-1.03 (m, 1H).

### EXAMPLE 12

### 4-{[2-(4-Cyanophenyl)-2-cyclopentylpropanoyl]oxy}-1,1-dimethylpiperidinium iodide

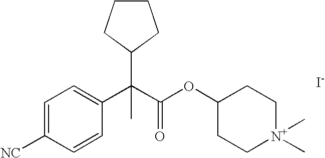

### a) Methyl 2-(4-cyanophenyl)-2-cyclopentylpropanoate

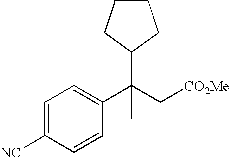

Methyl 2-(4-bromophenyl)-2-cyclopentylpropanoate (Example 11b) (0.312 g), zinc cyanide (0.07 g) and tetrakis(triphenylphosphine)palladium(0) (0.046 g) were dissolved in degassed N,N-dimethylformamide (3 mL) and the reaction mixture was heated at 80° C. under a nitrogen atmosphere. The mixture was cooled to room temperature, diluted with toluene and washed with 2N aqueous sodium hydroxide. The organic extract was washed with brine then dried over anhydrous magnesium sulphate, filtered and concentrated. The crude product was purified by flash chromatography on a silica column, eluting with 50% dichloromethane in ethyl acetate to yield the sub-titled compound (0.2 g).

¹H NMR (399.824 MHz, CDCl₃) δ 7.60 (d, 2H), 7.46 (d, 2H), 3.66 (s, 3H), 2.81-2.71 (m, 1H), 1.79-1.68 (m, 1H), 1.62-1.44 (m, 4H), 1.53 (s, 3H), 1.43-1.32 (m, 2H), 1.14-1.03 (m, 1H).

### b) 1-Methylpiperidin-4-yl 2-(4-cyanophenyl)-2-cyclopentylpropanoate

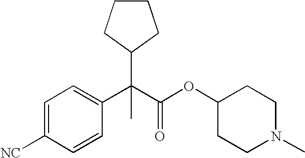

The sub-titled compound was prepared from methyl 2-(4-cyanophenyl)-2-cyclopentylpropanoate (Example 12a) (0.19 g) and 4-hydroxy-1-methylpiperidine (0.17 g) according to the method of Example 4d. The crude product was purified by flash chromatography on a silica column, eluting with 1% triethylamine in 5-10% methanol/dichloromethane to yield the sub-titled compound (0.2 g).

¹H NMR (399.824 MHz, CDCl₃) δ 7.60 (d, 2H), 7.48 (d, 2H), 4.84-4.76 (m, 1H), 2.85-2.74 (m, 1H), 2.47-2.35 (m, 2H), 2.28-2.17 (m, 2H), 2.22 (s, 3H), 1.87-1.79 (m, 2H), 1.79-1.70 (m, 1H), 1.68-1.46 (m, 5H), 1.52 (s, 3H), 1.46-1.34 (m, 2H), 1.15-1.03 (m, 1H), 0.91-0.80 (m, 1H).

### c) 4-{[2-(4-Cyanophenyl)-2-cyclopentylpropanoyl]oxy}-1,1-dimethylpiperidinium iodide

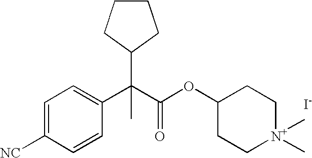

To a stirred solution of 1-methylpiperidin-4-yl 2-(4-cyanophenyl)-2-cyclopentylpropanoate (Example 12b) (0.16 g) in diethyl ether (4 mL) was added iodomethane (0.1 mL) and the reaction stirred for 2 hours. The resulting suspension was filtered and the colourless solid was washed with diethyl ether. On drying this gave the title compound (0.13 g).

m/e 355 (M)⁺

¹H NMR (399.826 MHz, DMSO) δ 7.82 (d, 2H), 7.58 (d, 2H), 4.95-4.88 (m, 1H), 3.44-3.35 (m, 2H), 3.26-3.15 (m, 2H), 3.09 (s, 3H), 3.07 (s, 3H), 2.87-2.76 (m, 1H), 2.15-2.04 (m, 2H), 1.88-1.74 (m, 2H), 1.72-1.62 (m, 1H), 1.57-1.42 (m, 4H), 1.51 (s, 3H), 1.42-1.31 (m, 2H), 1.16-1.03 (m, 1H).

### EXAMPLE 13

### 4-{[2-(3-Cyanophenyl)-2-cyclopentylpropanoyl]oxy}-1,1-dimethylpiperidinium iodide

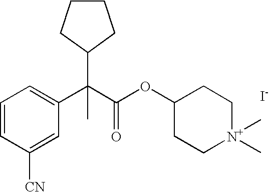

### a) Methyl 2-(3-cyanophenyl)-2-cyclopentylpropanoate

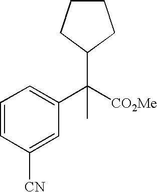

The sub-titled compound was prepared from methyl 2-(3-bromophenyl)-2-cyclopentylpropanoate (Example 10c) (0.93 g) according to the method of Example 12a. The crude product was purified by flash chromatography on a silica column, eluting with 50% dichloromethane in ethyl acetate to yield the sub-titled compound (0.63 g).

¹H NMR (399.824 MHz, CDCl₃) δ 7.66 (s, 1H), 7.60 (d, 1H), 7.53 (d, 1H), 7.42 (t, 1H), 3.67 (s, 3H), 2.82-2.72 (m, 1H), 1.79-1.68 (m, 1H), 1.62-1.45 (m, 4H), 1.53 (s, 3H), 1.44-1.31 (m, 2H), 1.14-1.02 (m, 1H).

### b) 1-Methylpiperidin-4-yl 2-(3-cyanophenyl)-2-cyclopentylpropanoate

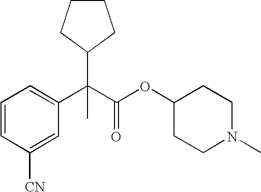

The sub-titled compound was prepared from methyl 2-(3-cyanophenyl)-2-cyclopentylpropanoate (Example 13a) (0.311 g) and 4-hydroxy-1-methylpiperidine (0.173 g) according to the method of Example 4d. The crude product was purified by flash chromatography on a silica column, eluting with 1% triethylamine in 5-10% methanol/dichloromethane to yield the sub-title compound (0.235 g).

¹H NMR (399.824 MHz, CDCl₃) δ 7.68 (s, 1H), 7.62 (d, 1H), 7.52 (d, 1H), 7.41 (t, 1H), 4.84-4.77 (m, 1H), 2.85-2.75 (m, 1H), 2.47-2.37 (m, 2H), 2.28-2.23 (m, 2H), 2.23 (s, 3H), 1.88-1.78 (m, 2H), 1.79-1.71 (m, 1H), 1.68-1.50 (m, 6H), 1.52 (s, 3H), 1.45-1.34 (m, 2H), 1.15-1.04 (m, 1H).

### c) 4-{[2-(3-Cyanophenyl)-2-cyclopentylpropanoyl]oxy}-1,1-dimethylpiperidinium iodide

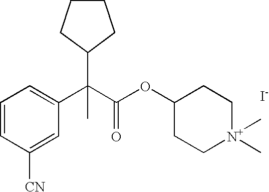

To a stirred solution of 1-methylpiperidin-4-yl 2-(3-cyanophenyl)-2-cyclopentylpropanoate (Example 13b) (0.22 g) in diethyl ether (2 mL) was added iodomethane (0.08 mL) and the reaction stirred for 2 hours. The resulting suspension was filtered and the colourless solid was washed with diethyl ether. On drying this gave the title compound (0.075 g).

m/e 355 (M)⁺

¹H NMR (399.826 MHz, DMSO) δ 7.85 (s, 1H), 7.74 (t, 2H), 7.56 (t, 1H), 4.96-4.89 (m, 1H), 3.44-3.35 (m, 2H), 3.27-3.18 (m, 2H), 3.10 (s, 3H), 3.07 (s, 3H), 2.91-2.80 (m, 1H), 2.16-2.05 (m, 2H), 1.87-1.76 (m, 2H), 1.72-1.61 (m, 1H), 1.57-1.41 (m, 4H), 1.52 (s, 3H), 1.41-1.30 (m, 2H), 1.14-1.03 (m, 1H).

### EXAMPLE 14

### 4-{[2-(3-Methylthiophenyl)-2-cyclopentylpropanoyl]oxy}-1,1-dimethylpiperidinium iodide

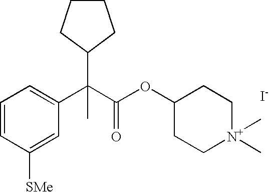

### a) 1-Methylpiperidin-4-yl 2-(3-methylthiophenyl)-2-cyclopentylpropanoate

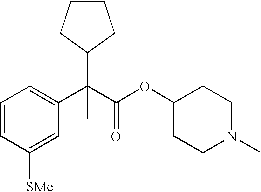

A mixture of 1-methylpiperidin-4-yl 2-(3-bromophenyl)-2-cyclopentylpropanoate (Example 10d) (1.47 g), sodium thiomethoxide (0.52 g), N,N-diisopropylethylamine (1.95 mL), 4,5-bis(diphenylphosphino)-9,9-dimethylxanthine (0.43 g) and tris(dibenzylideneacetone)dipalladium (0) (0.34 g) in 1,4-dioxane (15 mL) was heated under reflux for 4 hours. The reaction mixture was evaporated to dryness and the residue was purified by flash chromatography on a silica column, eluting with 1% triethylamine in ethyl acetate to yield the sub-title compound (1.13 g).

¹H NMR (399.824 MHz, CDCl₃) δ 7.29 (d, 2H), 7.18 (d, 2H), 4.83-4.75 (m, 1H), 2.88-2.77 (m, 1H), 2.47 (s, 3H), 2.49-2.38 (m, 2H), 2.28-2.20 (m, 2H), 2.23 (s, 3H), 1.87-1.78 (m, 2H), 1.78-1.67 (m, 2H), 1.69-1.34 (m, 7H), 1.48 (s, 3H), 1.17-1.06 (m, 1H)

### b) 4-{[2-(3-Methylthiophenyl)-2-cyclopentylpropanoyl]oxy}-1,1-dimethylpiperidinium iodide

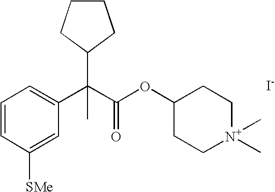

To a stirred solution of 1-methylpiperidin-4-yl 2-(3-methylthiophenyl)-2-cyclopentylpropanoate (Example 14a) (0.18 g) in diethyl ether (5 mL) was added iodomethane (0.06 mL) and the reaction stirred for 4 hours. The mixture was concentrated and the residue triturated with dry diethyl ether to give the title compound (0.16 g).

m/e 376 (M)⁺

¹H NMR (399.826 MHz, DMSO) δ 7.32 (d, 2H), 7.22 (d, 2H), 4.92-4.85 (m, 1H), 3.43-3.34 (m, 2H), 3.22-3.12 (m, 2H), 3.08 (s, 3H), 3.06 (s, 3H), 2.88-2.77 (m, 1H), 2.46 (s, 3H), 2.14-2.02 (m, 2H), 1.86-1.74 (m, 2H), 1.72-1.60 (m, 1H), 1.56-1.31 (m, 6H), 1.46 (s, 3H), 1.12-1.05 (m, 1H).

### EXAMPLE 15

### 4-{[2-(4-Methylthiophenyl)-2-cyclopentylpropanoyl]oxy}-1,1-dimethylpiperidinium iodide

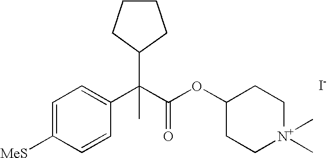

### a) 1-Methylpiperidin-4-yl 2-(4-methylthiophenyl)-2-cyclopentylpropanoate

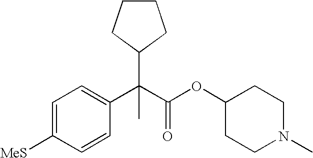

The sub-titled compound was prepared from 1-methylpiperidin-4-yl 2-(4-bromophenyl)-2-cyclopentylpropanoate (Example 11d) (0.6 g) according to the method of Example 14a. The crude product was purified by flash chromatography on a silica column, eluting with 1% triethylamine in ethyl acetate to yield the sub-title compound (0.23 g).

¹H NMR (399.824 MHz, CDCl₃) δ 7.29 (d, 2H), 7.18 (d, 2H), 4.83-4.75 (m, 1H), 2.88-2.77 (m, 1H), 2.47 (s, 3H), 2.49-2.38 (m, 2H), 2.28-2.20 (m, 2H), 2.23 (s, 3H), 1.87-1.78 (m, 2H), 1.78-1.67 (m, 2H), 1.69-1.34 (m, 7H), 1.48 (s, 3H), 1.17-1.06 (m, 1H)

### b) 4-{[2-(4-Methylthiophenyl)-2-cyclopentylpropanoyl]oxy}-1,1-dimethylpiperidinium iodide

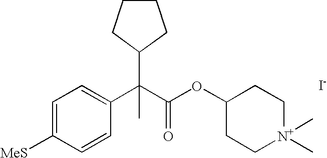

To a stirred solution of 1-methylpiperidin-4-yl 2-(4-methylthiophenyl)-2-cyclopentylpropanoate (Example 15a) (0.21 g) in diethyl ether (2 mL) was added iodomethane (0.07 mL) and the reaction stirred for 4 hours. The mixture was concentrated and the residue triturated with dry diethyl ether to give the title compound (0.25 g).

m/e 376 (M)⁺

¹H NMR (399.826 MHz, DMSO) δ 7.32 (d, 2H), 7.22 (d, 2H), 4.92-4.85 (m, 1H), 3.43-3.34 (m, 2H), 3.22-3.12 (m, 2H), 3.08 (s, 3H), 3.06 (s, 3H), 2.88-2.77 (m, 1H), 2.46 (s, 3H), 2.14-2.02 (m, 2H), 1.86-1.74 (m, 2H), 1.72-1.60 (m, 1H), 1.56-1.31 (m, 6H), 1.46 (s, 3H), 1.12-1.05 (m, 1H).

### EXAMPLE 16

### 4-{[2-(4-Methylsulfonylphenyl)-2-cyclopentylpropanoyl]oxy}-1,1-dimethylpiperidinium iodide

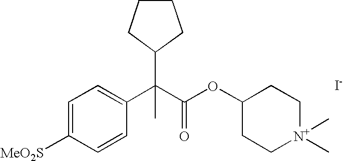

### a) 1-Methylpiperidin-4-yl 2-(4-methylsulfonylphenyl)-2-cyclopentylpropanoate

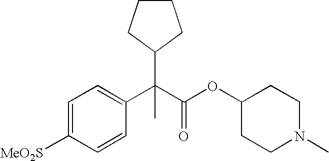

A solution of Oxone (1.85 g) in water (5 mL) was added to a solution of 1-methylpiperidin-4-yl 2-(4-methylthiophenyl)-2-cyclopentylpropanoate (Example 15a) (0.36 mg) in methanol (5 mL) at 0° C. The reaction mixture was allowed to warm to room temperature and after 1 hour was complete. The mixture was diluted with water and then extracted with dichloromethane. The organic extract was washed with brine then dried over anhydrous magnesium sulphate, filtered and concentrated. The crude product was purified by flash chromatography on a silica column, eluting with 1% triethylamine in 33% ethyl acetate/dichloromethane to yield the sub-title compound (0.2 g).

¹H NMR (399.824 MHz, CDCl₃) δ 7.94-7.83 (m, 2H), 7.63-7.53 (m, 2H), 4.87-4.77 (m, 1H), 3.06 (s, 3H), 2.90-2.77 (m, 1H), 2.52-2.37 (m, 2H), 2.32-2.17 (m, 2H), 2.23 (s, 3H), 1.92-1.80 (m, 2H), 1.81-1.36 (m, 5H), 1.55 (s, 3H), 1.35-1.22 (m, 2H), 1.19-1.05 (m, 2H), 0.93-0.79 (m, 1H).

### b) 4-{[2-(4-Methylsulfonylphenyl)-2-cyclopentylpropanoyl]oxy}-1,1-dimethylpiperidinium iodide

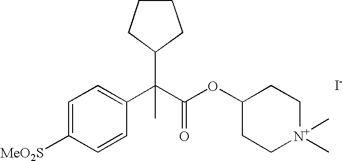

To a stirred solution of 1-methylpiperidin-4-yl 2-(4-methylsulfonylphenyl)-2-cyclopentylpropanoate (Example 16a) (0.08 g) in diethyl ether (3 mL) and acetonitrile (1 mL) was added iodomethane (0.05 mL) and the reaction stirred for 4 hours. The mixture was concentrated and the residue triturated with dry diethyl ether to give the title compound (0.093 g).

m/e 408 (M)⁺

¹H NMR (399.826 MHz, DMSO) δ 7.90 (d, 2H), 7.66 (d, 2H), 4.96-4.89 (m, 1H), 3.44-3.35 (m, 2H), 3.25-3.14 (m, 2H), 3.22 (s, 3H), 3.08 (s, 3H), 3.07 (s, 3H), 2.89-2.79 (m, 1H), 2.16-2.04 (m, 2H), 1.90-1.77 (m, 2H), 1.74-1.63 (m, 1H), 1.5.2 (s, 3H), 1.58-1.33 (m, 5H), 1.27-1.21 (m, 1H), 1.18-1.07 (m, 1H).

### EXAMPLE 17

### 4-{[2-(3-Methylsulfonylphenyl)-2-cyclopentylpropanoyl]oxy}-1,1-dimethylpiperidinium iodide

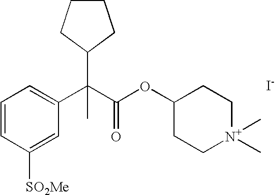

### a) 1-Methylpiperidin-4-yl 2-(3-methylsulfonylphenyl)-2-cyclopentylpropanoate

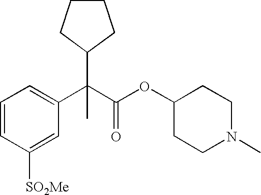

The sub-titled compound was prepared from 1-methylpiperidin-4-yl 2-(3-methylthiophenyl)-2-cyclopentylpropanoate (Example 14a) (0.36 g) according to the method of Example 16a. The crude product was purified by flash chromatography on a silica column, eluting with 1% triethylamine in 33% ethyl acetate/dichloromethane to yield the sub-title compound (0.19 g).

¹H NMR (399.824 MHz, CDCl₃) δ 7.98-7.95 (m, 1H), 7.83-7.79 (m, 1H), 7.70-7.65 (m, 1H), 7.52 (t, 1H), 4.86-4.79 (m, 1H), 3.49 (s, 3H), 3.04 (s, 3H), 2.89-2.79 (m, 1H), 2.50-2.38 (m, 2H), 2.29-2.22 (m, 2H), 2.23 (s, 3H), 1.89-1.80 (m, 2H), 1.80-1.70 (m, 1H), 1.70-1.36 (m, 8H), 1.17-1.05 (m, 1H).

### b) 4-{[2-(3-Methylsulfonylphenyl)-2-cyclopentylpropanoyl]oxy}-1,1-dimethylpiperidinium iodide

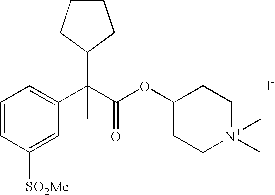

To a stirred solution of 1-methylpiperidin-4-yl 2-(3-methylsulfonylphenyl)-2-cyclopentylpropanoate (Example 17a) (0.18 g) in diethyl ether (5 mL) was added iodomethane (0.08 mL) and the reaction stirred for 4 hours. The mixture was concentrated and the residue triturated with dry diethyl ether to give the title compound (0.19 g).

m/e 408 (M)⁺

¹H NMR (399.826 MHz, DMSO) δ 7.90 (s, 1H), 7.85 (d, 1H), 7.77 (d, 1H), 7.65 (t, 1H), 4.98-4.91 (m, 1H), 3.44-3.35 (m, 2H), 3.27-3.16 (m, 2H), 3.25 (s, 3H), 3.09 (s, 3H), 3.06 (s, 3H), 2.95-2.85 (m, 1H), 2.17-2.06 (m, 2H), 1.87-1.77 (m, 2H), 1.74-1.65 (m, 1H), 1.59-1.44 (m, 4H), 1.55 (s, 3H), 1.43-1.34 (m, 2H), 1.17-1.06 (m, 1H).

### EXAMPLE 18

### 4-{[2-(4-Fluorophenyl)-2-cyclopentylpropanoyl]oxy}-1,1-dimethylpiperidinium iodide

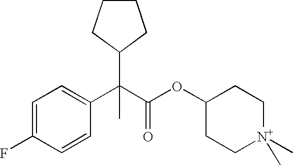

### a) Methyl (4-fluorophenyl)(cyclopentyl)acetate

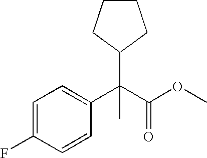

To a solution of the methyl (4-fluorophenyl)acetate (9.45 g) in tetrahydrofuran (80 mL) at −78° C. was added lithium diisopropylamide (37.5 mL of a 1.8M solution). After complete addition the reaction was maintained at this temperature for 30 minutes and a solution of bromocyclopentane (12.56 g) in tetrahydrofuran (20 mL) was added. The mixture was allowed to warm to room temperature and stirred overnight then heated at 50° C. for 2 hours. The reaction mixture was poured into concentrated ammonium chloride solution and extracted into diethyl ether. The combined organic extracts were dried over anhydrous magnesium sulphate, filtered and concentrated. The residue was purified on silica gel eluting with dichloromethane/iso-hexane (1/3) followed by a second purification using diethyl ether (5%) in iso-hexane to give the sub-titled compound (1.9 g).

¹H NMR (399.824 MHz, CDCl₃) δ 7.33-7.27 (m, 2H), 7.02-6.95 (m, 2H), 3.65 (s, 3H), 3.26 (d, 1H), 2.57-2.45 (m, 1H), 1.93-1.84 (m, 1H), 1.71-1.37 (m, 5H), 1.30-1.19 (m, 1H), 1.02-0.92 (m, 1H).

### b) Methyl 2-cyclopentyl-2-(4-fluorophenyl)propanoate

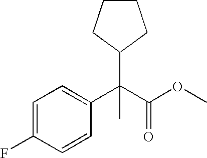

The sub-titled compound was prepared from methyl (4-fluorophenyl)cyclopentylacetate (Example 18a) (1.9 g) and iodomethane (0.8 mL) according to the method of Example 4c. On completion the reaction mixture was partitioned between saturated ammonium chloride solution and diethyl ether. The combined organic extracts were washed with water and brine then dried over anhydrous magnesium sulphate, filtered and concentrated. The crude product was purified by flash chromatography on a silica column, eluting with 33-50% dichloromethane in iso-hexane to yield the sub-titled compound (1.9 g).

¹H NMR (399.824 MHz, CDCl₃) δ 7.34-7.28 (m, 2H), 7.02-6.95 (m, 2H), 3.64 (s, 3H), 2.82-2.73 (m, 1H), 1.77-1.68 (m, 1H), 1.61-1.45 (m, 4H), 1.50 (s, 3H), 1.45-1.30 (m, 2H), 1.15-1.04 (m, 1H).

### c) 1-Methylpiperidin-4-yl 2-(4-fluorophenyl)-2-cyclopentylpropanoate

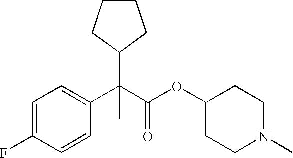

The sub-titled compound was prepared from methyl 2-(4-fluorophenyl)-2-cyclopentylpropanoate (Example 18b) (0.50 g) and 4-hydroxy-1-methylpiperidine (0.345 g) according to the method of Example 4d. The crude product was purified by flash chromatography on a silica column, eluting with 1% triethylamine, 5% methanol in dichloromethane to yield the sub-titled compound (0.43 g).

¹H NMR (399.824 MHz, CDCl₃) δ 7.37-7.31 (m, 2H), 7.01-6.94 (m, 2H), 4.82-4.76 (m, 1H), 2.87-2.77 (m, 1H), 2.47-2.35 (m, 2H), 2.28-2.19 (m, 2H), 2.22 (s, 3H), 1.86-1.78 (m, 2H), 1.78-1.69 (m, 2H), 1.67-1.33 (m, 7H), 1.49 (s, 3H), 1.16-1.05 (m, 1H).

### b) 4-{[2-(4-Fluorophenyl)-2-cyclopentylpropanoyl]oxy}-1,1-dimethylpiperidinium iodide

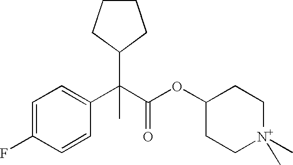

To a stirred solution of 1-methylpiperidin-4-yl 2-(4-fluorophenyl)-2-cyclopentylpropanoate (Example 18c) (0.4 g) in acetonitrile (5 mL) was added iodomethane (0.149 mL) and the reaction stirred for 2 hours. The mixture was concentrated and the residue triturated with dry diethyl ether to give the title compound (0.435 g).

¹H NMR (399.826 MHz, DMSO) δ 7.47-7.39 (m, 2H), 7.16 (t, 2H), 4.93-4.86 (m, 1H), 3.44-3.35 (m, 2H), 3.25-3.14 (m, 2H), 3.09 (s, 3H), 3.07 (s, 3H), 2.88-2.78 (m, 1H), 2.15-2.02 (m, 2H), 1.87-1.74 (m, 2H), 1.72-1.61 (m, 1H), 1.59-1.28 (m, 6H), 1.48 (s, 3H), 1.15-1.02 (m, 1H).

### EXAMPLE 19

### 4-{[2-(4-Chlorophenyl)-2-cyclopentylpropanoyl]oxy}-1,1-dimethylpiperidinium iodide

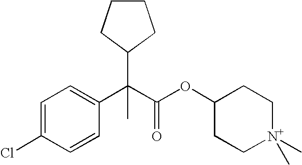

### a) Methyl (4-chlorophenyl)(cyclopentyl)acetate

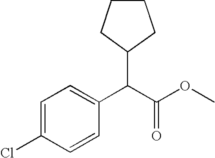

The sub title compound was prepared from methyl (4-chlorophenyl)acetate (9.23 g) and cyclopentyl bromide (8.04 mL) according to the method of Example 18a. The crude product was purified on silica gel eluting with diethyl ether (5%) in iso-hexane followed by a second purification using dichloromethane/iso-hexane (1/3) to give the sub-titled compound (3.6 g).

¹H NMR (399.824 MHz, CDCl₃) δ 7.27 (s, 4H), 3.65 (s, 3H), 3.25 (d, 1H), 2.56-2.45 (m, 1H), 1.93-1.84 (m, 1H), 1.72-1.52 (m, 3H), 1.53-1.37 (m, 2H), 1.29-1.19 (m, 1H), 1.03-0.92 (m, 1H).

### b) Methyl (4-chlorophenyl)cyclopentylpropanoate

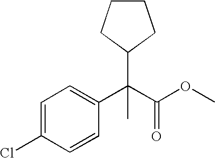

The sub-titled compound was prepared from methyl (4-chlorophenyl)cyclopentylacetate (Example 19a) (3.6 g) and iodomethane (1.42 mL) according to the method of Example 4c. On completion the reaction mixture was partitioned between saturated ammonium chloride solution and diethyl ether. The combined organic extracts were washed with water and brine then dried over anhydrous magnesium sulphate, filtered and concentrated. The residue was purified by flash chromatography on a silica column, eluting with 33-50% dichloromethane in iso-hexane to yield the sub-titled compound (3.4 g).

¹H NMR (399.824 MHz, CDCl₃) δ 7.27 (s, 4H), 3.64 (s, 3H), 2.81-2.71 (m, 1H), 1.78-1.68 (m, 1H), 1.61-1.45 (m, 4H), 1.49 (s, 3H), 1.45-1.30 (m, 2H), 1.15-1.03 (m, 1H).

### c) 1-Methylpiperidin-4-yl 2-(4-chlorophenyl)-2-cyclopentylpropanoate

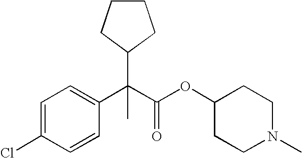

The sub-titled compound was prepared from methyl 2-(4-chlorophenyl)-2-cyclopentylpropanoate (Example 19b) (0.53 g) and 4-hydroxy-1-methylpiperidine (0.346 g) according to the method of Example 4d. The crude product was purified by flash chromatography on a silica column, eluting with 1% triethylamine, 5% methanol in dichloromethane to yield the sub-titled compound (0.40 g).

¹H NMR (399.824 MHz, CDCl₃) δ 7.37-7.23 (m, 4H), 4.85-4.75 (m, 1H), 2.87-2.76 (m, 1H), 2.51-2.36 (m, 2H), 2.31-2.18 (m, 2H), 2.23 (s, 3H), 1.89-1.78 (m, 2H), 1.79-1.31 (m, 9H), 1.49 (s, 3H), 1.17-1.04 (m, 1H).

### d) 4-{[2-(4-Chlorophenyl)-2-cyclopentylpropanoyl]oxy}-1,1-dimethylpiperidinium iodide

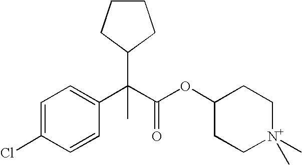

To a stirred solution of 1-methylpiperidin-4-yl 2-(4-chlorophenyl)-2-cyclopentylpropanoate (Example 19c) (0.35 g) in acetonitrile (5 mL) was added iodomethane (0.125 mL) and the reaction stirred for 1.5 hours. The mixture was concentrated and the residue dissolved in a mixture of propan-1-ol and ethyl acetate (1/1) then evaporated to dryness to give the title compound (0.240 g).

¹H NMR (399.826 MHz, DMSO) δ 7.40 (s, 4H), 4.94-4.86 (m, 1H), 3.44-3.34 (m, 2H), 3.25-3.15 (m, 2H), 3.09 (s, 3H), 3.07 (s, 3H), 2.86-2.76 (m, 1H), 2.14-2.03 (m, 2H), 1.87-1.74 (m, 2H), 1.72-1.61 (m, 1H), 1.56-1.43 (m, 4H), 1.47 (s, 3H), 1.41-1.30 (m, 2H), 1.14-1.04 (m, 1H).

### EXAMPLE 20

### 4-{[2-(3-Fluorophenyl)-2-cyclopentylpropanoyl]oxy}-1,1-dimethylpiperidinium iodide

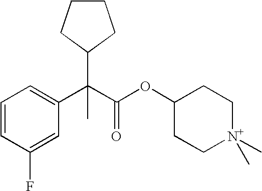

### a) Methyl (3-fluorophenyl)(cyclopentyl)acetate

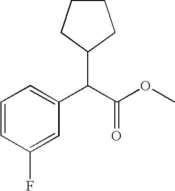

The sub title compound was prepared from methyl (3-fluorophenyl)acetate (10.0 g) and cyclopentyl bromide (9.57 mL) according to the method of Example 18a. The crude product was purified on silica gel eluting with diethyl ether (5%) in iso-hexane to give the sub-titled compound (6.8 g).

¹H NMR (399.824 MHz, CDCl₃) δ 7.30-7.23 (m, 1H), 7.13-7.06 (m, 2H), 6.98-6.91 (m, 1H), 3.66 (s, 3H), 3.28 (d, 1H), 2.58-2.46 (m, 1H), 1.94-1.85 (m, 1H), 1.73-1.53 (m, 3H), 1.54-1.39 (m, 2H), 1.31-1.19 (m, 1H), 1.06-0.95 (m, 1H).

### b) Methyl (3-fluorophenyl)cyclopentylpropanoate

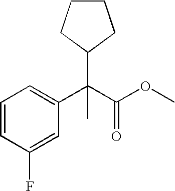

The sub-titled compound was prepared from methyl (3-fluorophenyl)cyclopentylacetate (Example 20a) (1.9 g) and iodomethane (0.8 mL) according to the method of Example 4c. On completion the reaction mixture was partitioned between saturated ammonium chloride solution and diethyl ether. The combined organic extracts were washed with water and brine then dried over anhydrous magnesium sulphate, filtered and concentrated. The crude product was purified by flash chromatography on a silica column, eluting with 20% dichloromethane in iso-hexane to yield the sub-titled compound (1.06 g).

¹H NMR (399.824 MHz, CDCl₃) δ 7.31-7.23 (m, 1H), 7.15-7.04 (m, 2H), 6.96-6.88 (m, 1H), 3.65 (s, 3H), 2.83-2.72 (m, 1H), 1.80-1.69 (m, 1H), 1.62-1.51 (m, 4H), 1.50 (s, 3H), 1.47-1.32 (m, 2H), 1.17-1.05 (m, 1H).

### c) 1-Methylpiperidin-4-yl 2-(3-fluorophenyl)-2-cyclopentylpropanoate

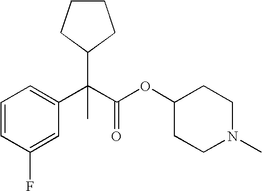

The sub-titled compound was prepared from methyl 2-(3-fluorophenyl)-2-cyclopentylpropanoate (Example 20b) (0.28 g) and 4-hydroxy-1-methylpiperidine (0.19 g) according to the method of Example 4d. The crude product was purified by flash chromatography on a silica column, eluting with 1% triethylamine, 2.5% methanol in dichloromethane to yield the sub-titled compound (0.18 g).

¹H NMR (399.824 MHz, CDCl₃) δ 7.28-7.22 (m, 1H), 7.16-7.12 (m, 1H), 7.12-7.07 (m, 1H), 6.94-6.88 (m, 1H), 4.84-4.76 (m, 1H), 2.85-2.75 (m, 1H), 2.47-2.35 (m, 2H), 2.29-2.20 (m, 2H), 2.23 (s, 3H), 1.88-1.79 (m, 2H), 1.79-1.35 (m, 9H), 1.50 (s, 3H), 1.18-1.07 (m, 1H).

### d) 4-{[2-(3-Fluorophenyl)-2-cyclopentylpropanoyl]oxy}-1,1-dimethylpiperidinium iodide

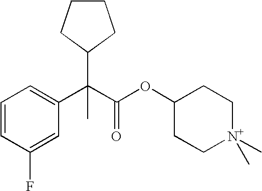

To a stirred solution of 1-methylpiperidin-4-yl 2-(3-fluorophenyl)-2-cyclopentylpropanoate (Example 20c) (0.15 g) in acetonitrile (2 mL) was added iodomethane (0.084 mL) and the reaction stirred for 3 hours. The mixture was concentrated and the residue triturated with dry diethyl ether to give the title compound (0.190 g).

¹H NMR (399.826 MHz, DMSO) δ 7.43-7.35 (m, 1H), 7.24-7.18 (m, 2H), 7.13-7.06 (m, 1H), 4.94-4.87 (m, 1H), 3.44-3.35 (m, 2H), 3.25-3.17 (m, 2H), 3.09 (s, 3H), 3.07 (s, 3H), 2.88-2.78 (m, 1H), 2.15-2.04 (m, 2H), 1.88-1.76 (m, 2H), 1.72-1.62 (m, 1H), 1.57-1.42 (m, 4H), 1.49 (s, 3H), 1.42-1.31 (m, 2H), 1.16-1.04 (m, 1H).

### EXAMPLE 21

### 4-{[2-Cyclopentyl-2-(2-thienyl)propanoyl]oxy}-1,1-dimethylpiperidinium iodide (Isomer 1)

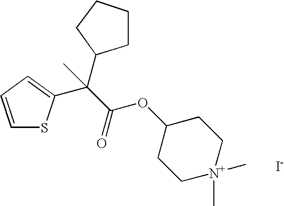

### a) Ethyl 2-cyclopentyl-2-(2-thienyl)propanoate (Enantiomer 1)

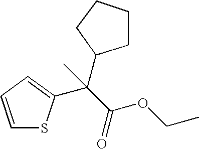

Racemic ethyl 2-cyclopentyl-2-(2-thienyl)propanoate as prepared in Example 8d (400 mg) was dissolved in isohexane:isopropanol (80:20, 5 mL) and resolved by chiral stationary phase HPLC using a Chiralcel OJ 10×250 mm column eluted isocratically with isohexane:isopropanol (90:10). Isomer 1 was the first enantiomer to elute from the column and gave a colourless oil (106 mg) after evaporation.

¹H NMR (400 MHz, CDCl₃) δ 7.17-7.20 (m, 1H), 6.95-6.98 (m, 1H), 6.91-6.94 (m, 1H), 4.10-4.18 (m, 2H), 2.78-2.88 (m, 1H), 1.64-1.73 (m, 1H), 1.46-1.63 (m, 8H), 1.30-1.41 (m, 1H), 1.19-1.29 (m, 1H), 1.24 (t, 3H).

### b) 1-Methylpiperidin-4-yl 2-cyclopentyl-2-(2-thienyl)propanoate (Isomer 1)

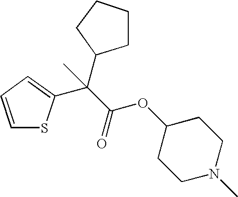

Prepared by the method of Example 8e using ethyl 2-cyclopentyl-2-(2-thienyl)propanoate, (Isomer 1) (Example 21a) (0.106 g) in place of racemic ethyl 2-cyclopentyl-2-(2-thienyl)propanoate. Yield 0.076 g.

m/e 322 (M+H)⁺

¹H NMR (400 MHz, CDCl₃) δ 7.16-7.20 (m, 1H), 6.95-6.98 (m, 1H), 6.90-6.94 (m, 1H), 4.78-4.85 (m, 1H), 2.80-2.90 (m, 1H), 2.41-2.56 (bm, 2H), 2.20-2.35 (bm, 2H), 2.26 (s, 3H), 1.80-1.93 (m, 2H), 1.62-1.75 (m, 4H), 1.46-1.62 (m, 4H), 1.56 (s, 3H), 1.31-1.44 (m, 1H), 1.19-1.29 (m, 1H).

### c) 4-{[2-Cyclopentyl-2-(2-thienyl)propanoyl]oxy}-1,1-dimethylpiperidinium iodide (Isomer 1)

Prepared by the method of Example 8f using 1-methylpiperidin-4-yl 2-cyclopentyl-2-(2-thienyl)propanoate (isomer 1) (Example 21b) (0.073 g) in place of racemic 1-methylpiperidin-4-yl 2-cyclopentyl-2-(2-thienyl)propanoate. Yield 0.098 g.

m/e 336 (M−I⁻)⁺

¹H NMR (400 MHz, DMSO-D₆) δ 7.44-7.47 (m, 1H), 7.06-7.09 (m, 1H), 6.97-7.01 (m, 1H), 4.85-4.92 (m, 1H), 3.35-3.46 (m, 2H), 3.14-3.30 (m, 2H), 3.12 (s, 3H), 3.07 (s, 3H), 2.82-2.92 (m, 1H), 2.05-2.19 (m, 2H), 1.78-1.92 (bm, 2H), 1.60-1.70 (m, 1H), 1.43-1.59 (m, 5H), 1.52 (s, 3H), 1.26-1.37 (m, 1H), 1.14-1.26 (m, 1H).

### EXAMPLE 22

### 4-{[2-Cyclopentyl-2-(2-thienyl)propanoyl]oxy}-1,1-dimethylpiperidinium iodide, (Isomer 2)

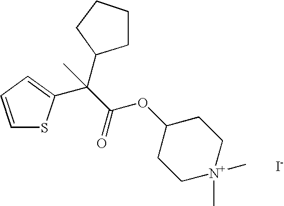

### a) Ethyl 2-cyclopentyl-2-(2-thienyl)propanoate (Isomer 2)

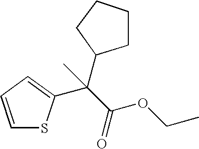

Enantiomer 2 was obtained as the second enantiomer to elute from the column in Example 21a. Yield 118 mg.

¹H NMR (400 MHz, CDCl₃) δ 7.17-7.20 (m, 1H), 6.95-6.98 (m, 1H), 6.91-6.94 (m, 1H), 4.10-4.18 (m, 2H), 2.78-2.88 (m, 1H), 1.64-1.73 (m, 1H), 1.46-1.62 (m, 8H), 1.30-1.41 (m, 1H), 1.19-1.29 (m, 1H), 1.24 (t, 3H).

### b) 1-Methylpiperidin-4-yl 2-cyclopentyl-2-(2-thienyl)propanoate (Isomer 2)

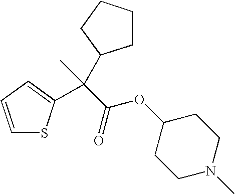

Prepared by the method of Example 8e using ethyl 2-cyclopentyl-2-(2-thienyl)propanoate, (Isomer 2) (Example 22a) (0.118 g) in place of racemic ethyl 2-cyclopentyl-2-(2-thienyl)propanoate. Yield 0.104 g.

m/e 322 (M+H)⁺.

¹H NMR (400 MHz, CDCl₃) δ 7.17-7.20 (m, 1H), 6.96-6.98 (m, 1H), 6.91-6.94 (m, 1H), 4.78-4.85 (m, 1H), 2.80-2.90 (m, 1H), 2.40-2.56 (bm, 2H), 2.20-2.35 (bm, 2H), 2.26 (s, 3H), 1.81-1.93 (m, 2H), 1.62-1.76 (m, 4H), 1.46-1.62 (m, 4H), 1.56 (s, 3H), 1.31-1.42 (m, 1H), 1.19-1.29 (m, 1H).

### c) 4-{[2-Cyclopentyl-2-(2-thienyl)propanoyl]oxy}-1,1-dimethylpiperidinium iodide (Isomer 2)

Prepared by the method of Example 8f using 1-methylpiperidin-4-yl 2-cyclopentyl-2-(2-thienyl)propanoate (Isomer 2) (Example 22b) (0.104 g) in place of racemic 1-methylpiperidin-4-yl 2-cyclopentyl-2-(2-thienyl)propanoate. Yield 0.144 g.

m/e 336 (M−I⁻)⁺.

¹H NMR (400 MHz, DMSO-D₆) δ 7.44-7.46 (m, 1H), 7.06-7.08 (m, 1H), 6.97-7.00 (m, 1H), 4.85-4.92 (m, 1H), 3.35-3.46 (m, 2H), 3.14-3.30 (m, 2H), 3.12 (s, 3H), 3.07 (s, 3H), 2.82-2.92 (m, 1H), 2.05-2.19 (m, 2H), 1.78-1.92 (bm, 2H), 1.60-1.70 (m, 1H), 1.43-1.59 (m, 5H), 1.52 (s, 3H), 1.26-1.37 (m, 1H), 1.14-1.26 (m, 1H).

### EXAMPLE 23

### 4-{[2-Cyclopentyl-2-(5-methyl-2-thienyl)propanoyl]oxy}-1,1-dimethylpiperidinium iodide (Isomer 1)

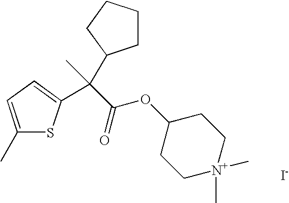

### a) 2-Cyclopentyl-2-(5-methyl-2-thienyl)propanoic acid (racemate)

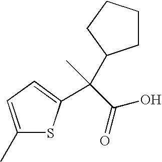

A solution of ethyl 2-cyclopentyl-2-(5-methyl-2-thienyl)propanoate (Example 9a) (2.03 g) in ethanol (40 mL) containing 2 molar aqueous potassium hydroxide (8 mL) was heated at reflux for 48 hours. The cooled reaction mixture was concentrated to remove ethanol, acidified with 2 molar hydrochloric acid and extracted twice with diethyl ether. The is combined organic extracts were washed with brine, dried (MgSO₄) and concentrated to give an orange oil. Purification was by preparative HPLC using a Waters Xterra MS C8 column eluted with 0.2% aqueous trifluoroacetic acid and acetonitrile. Yield 1.12 g.

¹H NMR (400 MHz, CDCl₃) δ 6.78 (d, 1H), 6.57-6.59 (m, 1H), 2.79 (quin, 1H), 2.43 (s, 3H), 1.68-1.79 (m, 1H), 1.46-1.65 (m, 5H), 1.53 (s, 3H), 1.30-1.42 (m, 1H), 1.19-1.30 (m, 1H).

### b) (3aS,6R,7aR)-1-[2-cyclopentyl-2-(5-methyl-2-thienyl)propanoyl]-8,8-dimethylhexahydro-3a,6-methano-2,1-benzisothiazole 2,2-dioxide (Isomer 1)

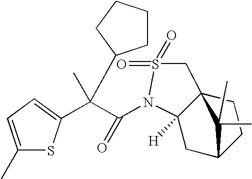

Oxalyl chloride (0.78 mL) was added to a solution of 2-cyclopentyl-2-(5-methyl-2-thienyl)propanoic acid (Example 23a) (1.07 g) in dichloromethane (20 mL) and N,N-dimethylformamide (0.1 mL). The resulting mixture was stirred at room temperature for 1 hour and then concentrated to afford the crude acid chloride as a yellow residue.

Simultaneously, n-butyl lithium solution in hexanes (2.0 M, 2.7 mL) was added to a solution of (2R)-bornane-10,2-sultam (1.15 g) in tetrahydrofuran (20 mL), pre-cooled in ice-water. The cooling bath was removed and the colourless solution was stirred at room temperature for 1 hour. The resulting yellow solution was cooled again in ice-water and treated with a suspension of the acid chloride generated above in tetrahydrofuran (35 mL), added portionwise over 10 minutes. The cooling bath was removed and the mixture was stirred at room temperature over 48 hours. The yellow solution was then poured into 2 molar hydrochloric acid and extracted twice with diethyl ether. The combined organic extracts were dried (MgSO₄) and concentrated onto flash silica. The resulting powder was purified by flash chromatography on silica eluted with 20% diethyl ether in isohexane. Isomer 1 was the first diastereomer of the product mixture to elute from the column and was obtained as an off-white solid (0.85 g).

m/e 436 (M+H)⁺

¹H NMR (400 MHz, CDCl₃) δ 6.74 (d, 1H), 6.55-6.57 (m, 1H), 4.00-4.05 (m, 1H), 3.35 (s, 2H), 3.06 (quin, 1H), 2.42 (s, 3H), 1.86-1.95 (m, 1H), 1.69-1.86 (m, 5H), 1.71 (s, 3H), 1.45-1.66 (m, 5H), 1.24-1.39 (m, 4H), 0.92 (s, 3H), 0.88 (s, 3H).

### c) 2-Cyclopentyl-2-(5-methyl-2-thienyl)propanoic acid (Isomer 1)

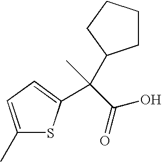

A solution of (3aS,6R,7aR)-1-[2-cyclopentyl-2-(5-methyl-2-thienyl)propanoyl]-8,8-dimethylhexahydro-3a,6-methano-2,1-benzisothiazole 2,2-dioxide, (Isomer 1) (Example 23b) (0.79 g) in tetrahydrofuran (24 mL) and water (8 mL) was treated with a 30% solution of hydrogen peroxide in water (0.95 mL) and lithium hydroxide monohydrate (93 mg). The resulting mixture was stirred at room temperature overnight and then quenched by the addition of a 10% aqueous solution of sodium metabisulphite. After being stirred for 15 minutes, the mixture was basified with 10% aqueous sodium hydroxide and extracted twice with dichloromethane. The chlorinated phases were discarded whilst the aqueous phase was acidified with 2 molar hydrochloric acid and extracted three times with diethyl ether. The combined organic extracts were washed with brine, then dried (MgSO₄) and concentrated onto flash silica. The resulting powder was purified by flash chromatography on silica eluted with acetic acid:diethyl ether:isohexane (1:30:69) to afford the subtitled compound as a white solid (0.40 g).

m/e 238 (M)⁺

¹H NMR (400 MHz, CDCl₃) δ 6.77 (d, 1H), 6.57-6.59 (m, 1H), 2.79 (quin, 1H), 2.43 (s, 3H), 1.68-1.79 (m, 1H), 1.46-1.65 (m, 5H), 1.53 (s, 3H), 1.31-1.42 (m, 1H), 1.19-1.30 (m, 1H).

### d) 1-Methylpiperidin-4-yl 2-cyclopentyl-2-(5-methyl-2-thienyl)propanoate (Isomer 1)

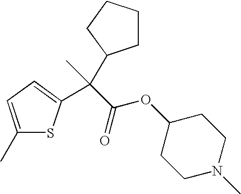

Oxalyl chloride (0.066 mL) was added to a solution of 2-cyclopentyl-2-(5-methyl-2-thienyl)propanoic acid, (Isomer 1) (Example 23c) (0.15 g) in dichloromethane (3 μL) and N,N-dimethylformamide (0.01 mL). The resulting mixture was stirred at room temperature for 1 hour and then concentrated to afford the crude acid chloride as a yellow residue.

Simultaneously, n-butyl lithium solution in hexanes (2.0 M, 0.38 mL) was added to a solution of 4-hydroxy-1-methylpiperidine (0.087 g) in tetrahydrofuran (3 mL), pre-cooled in ice-water. The cooling bath was removed and the colourless solution was stirred at room temperature for 1 hour. The resulting cloudy solution was then treated with a solution of the acid chloride generated above in tetrahydrofuran (4.5 mL), added portionwise over 1 minute. The mixture was stirred at room temperature for 1 hour and then concentrated onto flash silica. The resulting powder was purified by flash chromatography on silica eluted with triethylamine:ethanol:isohexane (1:5:94) to afford the subtitled compound as a pale yellow oil (0.13 g).

m/e 336 (M+H)⁺

¹H NMR (400 MHz, CDCl₃) δ 6.72 (d, 1H), 6.54-6.56 (m, 1H), 4.76-4.84 (m, 1H), 2.80 (quint, 1H), 2.44-2.57 (bm, 2H), 2.42 (s, 3H), 2.22-2.34 (bm, 2H), 2.27 (s, 3H), 1.81-1.92 (m, 2H), 1.62-1.76 (m, 4H), 1.46-1.62 (m, 4H), 1.51 (s, 3H), 1.29-1.40 (m, 1H), 1.18-1.29 (m, 1H).

### e) 4-{[2-Cyclopentyl-2-(5-methyl-2-thienyl)propanoyl]oxy}-1,1-dimethylpiperidinium iodide (Isomer 1)

Prepared by the method of Example 1g using 1-methylpiperidin-4-yl 2-cyclopentyl-2-(5-methyl-2-thienyl)propanoate (Isomer 1) (Example 23d) (0.13 g) in place of 1-methylpiperidin-4-yl (2S)-2-cyclopentyl-2-phenylpropanoate. Yield 0.16 g.

m/e 350 (M−I⁻)⁺

¹H NMR (400 MHz, DMSO-D₆) δ 6.83 (d, 1H), 6.64-6.66 (m, 1H), 4.84-4.91 (m, 1H), 3.36-3.47 (m, 1H), 3.15-3.34 (m, 2H), 3.12 (s, 3H), 3.08 (s, 3H), 2.76-2.86 (m, 2H), 2.39 (s, 3H), 2.05-2.18 (m, 2H), 1.79-1.92 (bm, 2H), 1.58-1.68 (m, 1H), 1.42-1.58 (m, 5H), 1.46 (s, 3H), 1.13-1.35 (m, 2H).

### EXAMPLE 24

### 4-{[2-Cyclopentyl-2-(5-methyl-2-thienyl)propanoyl]oxy}-1,1-dimethylpiperidinium iodide (Isomer 2)

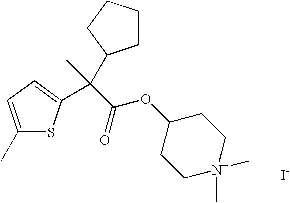

### a) (3aS,6R,7aR)-1-[2-cyclopentyl-2-(5-methyl-2-thienyl)propanoyl]-8,8-dimethylhexahydro-3a,6-methano-2,1-benzisothiazole 2,2-dioxide, (Isomer 2)

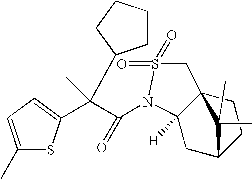

Isomer 2 was obtained as the second diastereomer to elute from the column in Example 23b. Yield 0.37 g.

m/e 436 (M+H)⁺

¹H NMR (400 MHz, CDCl₃) δ 6.72 (d, 1H), 6.57-6.59 (m, 1 μl), 4.00-4.05 (m, 1H), 3.26-3.36 (m, 2H), 2.93 (quin, 1H), 2.43 (s, 3H), 1.99-2.06 (m, 1H), 1.71-1.90 (m, 5H), 1.70 (s, 3H), 1.13-1.62 (m, 9H), 1.11 (s, 3H), 0.92 (s, 3H).

### b) 2-Cyclopentyl-2-(5-methyl-2-thienyl)propanoic acid (Isomer 2)

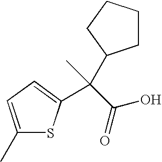

Prepared by the method of Example 23c using (3aS,6R,7aR)-1-[2-cyclopentyl-2-(5-methyl-2-thienyl)propanoyl]-8,8-dimethylhexahydro-3a,6-methano-2,1-benzisothiazole 2,2-dioxide, (Isomer 2) (Example 24a) (0.31 g) in place of (3aS,6R,7aR)-1-[2-cyclopentyl-2-(5-methyl-2-thienyl)propanoyl]-8,8-dimethylhexahydro-3a,6-methano-2,1-benzisothiazole 2,2-dioxide, (Isomer 1). Yield 0.14 g.

m/e 238 (M+H)⁺

¹H NMR (400 MHz, CDCl₃) δ 6.77 (d, 1H), 6.57-6.59 (m, 1H), 2.79 (quin, 1H), 2.43 (s, 3H), 1.68-1.79 (m, 1H), 1.46-1.65 (m, 5H), 1.53 (s, 3H), 1.31-1.44 (m, 1H), 1.19-1.30 (m, 1H).

### c) 1-Methylpiperidin-4-yl 2-cyclopentyl-2-(5-methyl-2-thienyl)propanoate (Isomer 2)

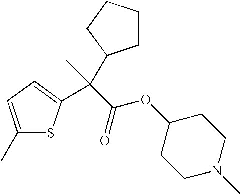

Prepared by the method of Example 23d using 2-cyclopentyl-2-(5-methyl-2-thienyl)propanoic acid (Isomer 2) (Example 24b) (0.14 g) in place of 2-cyclopentyl-2-(5-methyl-2-thienyl)propanoic acid (Isomer 1). Yield 0.12 g.

m/e 336 (M+H)⁺

¹H NMR (400 MHz, CDCl₃) δ 6.72 (d, 1H), 6.54-6.56 (m, 1H), 4.76-4.84 (m, 1H), 2.80 (quin, 1H), 2.44-2.57 (bm, 2H), 2.42 (s, 3H), 2.19-2.35 (bm, 2H), 2.26 (s, 3H), 1.81-1.92 (m, 2H), 1.61-1.76 (m, 4H), 1.46-1.61 (m, 4H), 1.51 (s, 3H), 1.29-1.40 (m, 1H), 1.18-1.29 (m, 1H).

### d) 4-{[2-Cyclopentyl-2-(5-methyl-2-thienyl)propanoyl]oxy}-1,1-dimethylpiperidinium iodide (Isomer 2)

Prepared by the method of Example 1g using 1-methylpiperidin-4-yl 2-cyclopentyl-2-(5-methyl-2-thienyl)propanoate, (Enantiomer 2) (Example 24c) (0.12 g) in place of 1-methylpiperidin-4-yl (2S)-2-cyclopentyl-2-phenylpropanoate. Yield 0.15 g.

m/e 350 (M−I⁻)⁺

¹H NMR (400 MHz, DMSO-D₆) δ 6.83 (d, 1H), 6.64-6.66 (m, 1H), 4.84-4.91 (m, 1H), 3.36-3.47 (m, 1H), 3.15-3.34 (m, 2H), 3.12 (s, 3H), 3.08 (s, 3H), 2.76-2.86 (m, 2H), 2.39 (s, 3H), 2.05-2.18 (m, 2H), 1.79-1.92 (bm, 2H), 1.58-1.68 (m, 1H), 1.42-1.58 (m, 5H), 1.46 (s, 3H), 1.13-1.36 (m, 2H).

### EXAMPLE 25

### 4-{[2-(5-Chloro-2-thienyl)-2-cyclobutylpropanoyl]oxy}-1,1-dimethylpiperidinium iodide (Isomer 1)

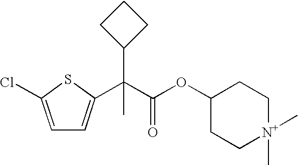

### a) (5-Chloro-2-thienyl)(cyclobutyl)methanone

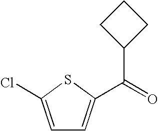

To a solution of N-methoxy-N-methylcyclobutanecarboxamide (3.4 g) in anhydrous tetrahydrofuran (25 mL) at 0° C. under an atmosphere of nitrogen was added 5-chloro-2-thienylmagnesium bromide (0.5M in tetrahydrofuran, 50 mL). The mixture was allowed to warm to room temperature over 24 hours and then poured into hydrochloric acid (1M aqueous) and extracted with diethyl ether (3×25 mL). The combined organic extracts were dried over anhydrous magnesium sulphate, filtered and concentrated. The crude product was purified by flash chromatography on a silica column, eluting with 10% ethyl acetate in iso-hexane to yield the sub-titled compound (3 g).

¹H NMR (399.824 MHz, CDCl₃) δ 7.38 (d, 1H), 6.93 (d, 1H), 3.81 (dquint, 1H), 2.48-2.37 (m, 2H), 2.31-2.21 (m, 2H), 2.14-2.01 (m, 1H), 1.99-1.87 (m, 1H).

### b) Methyl (5-chloro-2-thienyl)(cyclobutyl)acetate

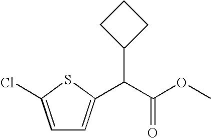

To DMSO (5 μL) at room temperature under an atmosphere of nitrogen was added NaH (60% dispersion in mineral oil, 671 mg). The suspension was stirred for 5 minutes and then heated to 70° C. for 1 hour. The mixture was cooled to room temperature, diluted with anhydrous tetrahydrofuran (12 mL) and cooled to −4° C. A solution of trimethylsulfonium iodide in anhydrous DMSO was added over 1 minute. The mixture was stirred for 10 minutes and then (5-chloro-2-thienyl)(cyclobutyl)methanone (Example 25a, 2.8 g) in tetrahydrofuran (5 mL) was added. The mixture was stirred at −4° C. for 15 hours and then allowed to warm up to room temperature overnight. The mixture was poured into saturated aqueous ammonium chloride solution and extracted with diethyl ether (3×15 mL). The combined organic extracts were dried over anhydrous magnesium sulphate, filtered and concentrated. Of the 2.5 g of crude product formed 1.5 g was dissolved in anhydrous tetrahydrofuran and IrCl₃ (30 mg) added. The mixture was stirred at room temperature for 30 minutes and then filtered and evaporated to dryness. The resulting oil (1 g) which was re-dissolved in a mixture of dichloromethane (14 mL) and water (2 mL) and 1-methyl-1-cyclohexene (3 g) added. The solution was cooled to 5° C. and aqueous sulphamic acid solution (1M, 2.5 mL) added dropwise. To the rapidly stirred mixture at 5° C. was added aqueous sodium chlorite solution (1M, 16 mL) over 30 minutes. The mixture was allowed to warm to room temperature and poured into saturated sodium bicarbonate solution (10 mL) and extracted with dichloromethane (2×20 mL). The aqueous phase was adjusted to pH 1 with dilute hydrochloric acid and extracted with diethyl ether (3×25 mL). The combined ethereal extract was dried with anhydrous magnesium sulphate, filtered and concentrated. The resulting crude product (740 mg) was dissolved in methanol (2 mL) and tetrahydrofuran (10 mL) and treated with trimethylsilyldiazomethane solution (2M in tetrahydrofuran, 2 mL). After 30 minutes at room temperature the mixture was poured into 1M aqueous hydrochloric acid and extracted with diethyl ether (3×15 mL). The combined ethereal extract was dried with anhydrous magnesium sulphate, filtered and concentrated. The crude product was purified by flash chromatography on a silica column, eluting with 5% ethyl acetate in iso-hexane to yield the sub-titled compound (300 mg).

¹H NMR (399.824 MHz, CDCl₃) δ 6.73 (d, 1H), 6.67 (d, 1H), 3.72 (d, 1H), 3.69 (s, 3H), 2.90-2.76 (m, 1H), 2.16-1.62 (m, 6H).

### c) Methyl 2-(5-chloro-2-thienyl)-2-cyclobutylpropanoate

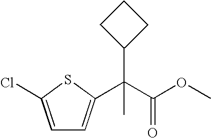

To a solution of dry diisopropylamine (320 mg) in tetrahydrofuran (3 mL) at −78° C. was added n-butyllithium (1.6M, 1.93 mL). The mixture was allowed to warm to −30° C. for 15 minutes and then re-cooled to −78° C. whereupon methyl (5-chloro-2-thienyl)(cyclobutyl)acetate (Example 25b, 300 mg) in tetrahydrofuran (3 mL) was added. The mixture was warmed to −30° C. and stirred for 30 minutes before iodomethane (450 mg) was added and the mixture allowed to warm to 10° C. over 2 hours. The reaction was poured into aqueous ammonium chloride solution and extracted into diethyl ether (2×30 mL). The combined organic extracts were dried over anhydrous magnesium sulphate, filtered and concentrated to give the sub-titled compound (300 mg) as an oil.

¹H NMR (400 MHz, CDCl₃) [symbol] 6.74 (d, 1H), 6.66 (d, 1H), 3.68 (s, 3H), 3.11-2.93 (m, 1H), 1.99-1.52 (m, 9H).

### d) 1-Methylpiperidin-4-yl 2-(5-chloro-2-thienyl)-2-cyclobutylpropanoate

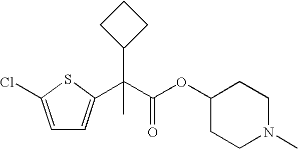

The sub-titled compound was prepared from methyl 2-(5-chloro-2-thienyl)-2-cyclobutylpropanoate (Example 25c) (75 mg) and 4-hydroxy-1-methylpiperidine (200 mg) according to the method of Example 4d. The crude product was purified by flash chromatography on a silica column, eluting with 1% triethylamine in ethyl acetate/iso-hexane (1/1) to yield the sub-titled compound (60 mg).

m/e 342 (M+H)⁺

¹H NMR (400 MHz, CDCl₃) [symbol] 6.74 (d, 1H), 6.67 (d, 1H), 4.82 (septet, 1H), 3.01 (quintet, 1H), 2.57-2.44 (m, 2H), 2.34-2.23 (m, 5H), 1.98-1.64 (m, 10H), 1.56 (s, 3H).

### e) 4-{[2-(5-Chloro-2-thienyl)-2-cyclobutylpropanoyl]oxy}-1,1-dimethylpiperidinium iodide (Isomer 1)

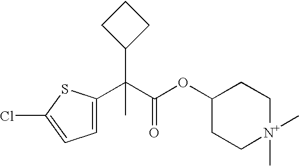

Racemic 1-methylpiperidin-4-yl 2-(5-chloro-2-thienyl)-2-cyclobutylpropanoate (Example 25d, 60 mg) was separated by Reversed Phase HPLC using a (R,R) Whelk column and ethanol/isohexane/diethylamine 10/90/0.1 as mobile phase. The first eluted isomer was evaporated to dryness and treated with iodomethane (0.3 mL) in acetonitrile (1 mL). The resulting mixture was stirred overnight, concentrated and triturated with diethyl ether to give the titled compound (35 mg).

m/e 356.1 (M⁺)

¹H NMR (400 MHz, DMSO-D₆) δ 7.00 (d, 1H), 6.88 (d, 1H), 4.94-4.89 (m, 1H), 3.47-3.40 (m, 2H), 3.34-3.26 (m, 2H), 3.14 (s, 3H), 3.08 (s, 3H), 3.07-3.00 (m, 1H), 2.18-2.09 (m, 2H), 1.93-1.75 (m, 7H), 1.69-1.60 (m, 1H), 1.55 (s, 3H).

### EXAMPLE 26

### 4-{[2-(5-Chloro-2-thienyl)-2-cyclobutylpropanoyl]oxy}-1,1-dimethylpiperidinium iodide (Isomer 2)

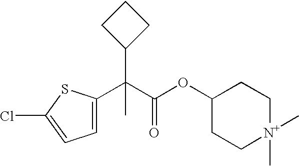

The titled compound was prepared according to the method of Example 25(e) using the second eluting compound to afford 25 mg of solid.

m/e 356.1 (M⁺)

¹H NMR (400 MHz, DMSO-D₆) δ 6.97 (d, 1H), 6.86 (d, 1H), 4.90 (septet, 1H), 3.33-3.20 (m, 2H), 3.41-3.36 (m, 2H), 3.12 (s, 3H), 3.07 (s, 3H), 3.04-2.96 (m, 1H), 2.18-2.06 (m, 2H), 1.92-1.75 (m, 7H), 1.64-1.58 (m, 1H), 1.54 (s, 3H).

### EXAMPLE 27

### 4-[(2-Cyclopropyl-2-phenylpropanoyl)oxy]-1,1-dimethylpiperidinium iodide (Isomer 1)

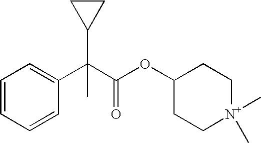

### a) (4R)-4-Benzyl-3-(2-cyclopropyl-2-phenylpropanoyl)-1,3-oxazolidin-2-one

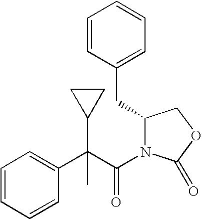

A solution of 2-cyclopropyl-2-phenylpropanoic acid (prepared according to the method outlined in Journal of Organic Chemistry (1989), 54(21), 5054-63, 0.71 g) in dichloromethane (10 mL) was treated with oxalyl chloride (1 mL) and N,N-dimethylformamide (10 mg) and the resultant mixture stirred at room temperature for 2 hours. The solvent was removed under reduced pressure and the residue azeotroped twice with dichloromethane. The residue was dissolved in anhydrous tetrahydrofuran (2 mL) and added dropwise over 5 minutes to a solution of (R)-4-benzyl-2-oxazolidinone (0.66 g) in anhydrous tetrahydrofuran (10 mL) at −78° C., which had been pre-treated at −78° C. with a 1.6 molar solution of n-butyllithium in hexanes (2.3 mL). The reaction mixture was stirred at −78° C. for 30 minutes and then at room temperature for 1 hour. At the end of this time the mixture was partitioned between ethyl acetate and saturated aqueous ammonium chloride solution, the organic layer was washed with aqueous brine, dried and evaporated under reduced pressure. Purification was by flash chromatography on a silica column, eluting with 12% tetrahydrofuran in isohexane to yield isomer 1 (0.31 g) and isomer 2 (0.35 g).

Isomer 1

¹H NMR (400 MHz, DMSO-D₆) δ 7.34-7.25 (m, 7H), 7.18-7.14 (m, 3H), 4.83-4.79 (m, 1H), 4.26 (t, 1H), 4.11 (q, 1H), 3.18 (q, 1H), 2.91 (q, 1H), 1.78-1.76 (m, 1H), 1.31 (s, 3H), 0.52-0.48 (m, 1H), 0.41-0.38 (m, 1H), 0.30-0.28 (m, 1H), 0.07-0.04 (m, 1H).

Isomer 2

¹H NMR (400 MHz, DMSO-D₆) δ 7.33-7.24 (m, 9H), 7.17-7.13 (m, 1H), 4.86-4.82 (m, 1H), 4.26 (t, 1H), 4.11 (q, 1H), 3.06 (q, 1H), 2.99-2.93 (m, 1H), 2.21-2.17 (m, 1H), 1.04 (s, 3H), 0.72-0.68 (m, 1H), 0.62-0.58 (m, 1H), 0.54-0.48 (m, 1H), 0.29-0.25 (m, 1H).

### b) 2-Cyclopropyl-2-phenylpropanoic acid (Isomer 1)

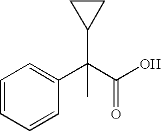

To a solution of 4-benzyl-3-(2-cyclopropyl-2-phenyl-propionyl)-oxazolidin-2-one (isomer 1) (0.31 g) in a mixture of tetrahydrofuran (30 mL) and water (10 mL) stirred at 0° C. was added, dropwise a 35 wt. % solution of hydrogen peroxide in water (0.79 mL). The mixture was then treated dropwise with a solution of lithium hydroxide monohydrate (76 mg) in water (1 mL) followed by stirring for 5 hours at room temperature. At the end of this time the reaction mixture was cooled in an ice bath and treated with a 1 molar solution of sodium sulphite in water (50 mL). The mixture was partitioned between dichloromethane and water. The aqueous layer was cooled and acidified by dropwise addition of dilute hydrochloric acid, the resultant precipitate was extracted into ethyl acetate and the organic layer was dried over anhydrous magnesium sulphate and evaporated under reduced pressure to yield the sub-titled compound (0.13 g).

m/e 189 (M−H)⁻

### c) 1-Methylpiperidin-4-yl 2-cyclopropyl-2-phenylpropanoate (Isomer 1)

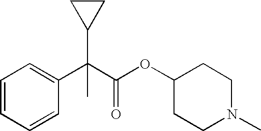

A solution of 2-cyclopropyl-2-phenylpropanoic acid (isomer 1) (130 mg) in dichloromethane (7 mL) was treated with oxalyl chloride (0.5 mL) and N,N-dimethylformamide (10 mg) and the resultant mixture stirred at room temperature for 2 hours. The solvent was removed under reduced pressure and the residue azeotroped three times with dichloromethane. The residue was dissolved in dichloromethane (4 mL) and was treated with 4-hydroxy-1-methylpiperidine (242 mg) and the reaction mixture was heated at 40° C. for 18 hours. The reaction mixture was partitioned between dichloromethane and water, the organic layer was dried over anhydrous magnesium sulphate and evaporated under reduced pressure. The crude product was purified by flash chromatography on a silica column, eluting with 1% triethylamine and 3% methanol in dichloromethane to yield the sub-titled compound (90 mg).

m/e 288 (M+H)⁺

¹H NMR (400 MHz, DMSO-D₆) δ 7.36-7.31 (m, 4H), 7.26-7.22 (m, 1H), 4.75-4.72 (m, 1H), 2.35-2.20 (m, 2H), 2.20-2.10 (m, 2H), 2.08 (s, 3H), 1.77-1.70 (m, 2H), 1.52-1.43 (m, 3H), 1.17 (s, 3H), 0.59-0.56 (m, 1H), 0.48-0.45 (m, 1H), 0.35-0.28 (m, 2H).

### d) 4-[(2-Cyclopropyl-2-phenylpropanoyl)oxy]-1,1-dimethylpiperidinium iodide (Isomer 1)

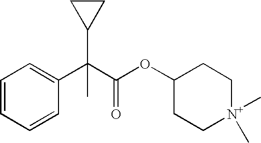

A solution of 1-methylpiperidin-4-yl 2-cyclopropyl-2-phenylpropanoate (Isomer 1) (Example 27c, 82 mg) in acetonitrile (2 mL) was treated with a solution of methyl iodide (53 mg) in dichloromethane (1 mL). The mixture was allowed to stand at room temperature for 90 minutes. At the end of this time the solvents were evaporated under reduced pressure and the residue triturated with diethyl ether to yield the titled compound as a white solid (120 mg).

m/e 302 (M)⁺

¹H NMR (400 MHz, DMSO-D₆) δ 7.41-7.33 (m, 4H), 7.29-7.26 (m, 1H), 4.96-4.93 (m, 1H), 3.43-3.35 (m, 2H), 3.25-3.13 (m, 2H), 3.07 (s, 3H), 3.05 (s, 3H), 2.15-2.07 (m, 2H), 1.90-1.75 (m, 2H), 1.58-1.53 (m, 1H), 1.20 (s, 3H), 0.61-0.58 (m, 1H), 0.51-0.47 (m, 1H), 0.39-0.30 (m, 2H).

### EXAMPLE 28

### 4-[(2-Cyclopropyl-2-phenylpropanoyl)oxy]-1,1-dimethylpiperidinium iodide (Isomer 2)

### a) 2-Cyclopropyl-2-phenylpropanoic acid (Isomer 2)

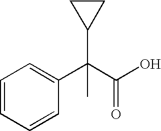

The sub-titled compound was prepared from (4R)-4-benzyl-3-(2-cyclopropyl-2-phenylpropanoyl)-1,3-oxazolidin-2-one (isomer 2) prepared as described in Example 27a (0.34 g) using the method of Example 27b. Yield 0.16.

m/e 189 (M−H)⁺

### b) 1-Methylpiperidin-4-yl 2-cyclopropyl-2-phenylpropanoate (Isomer 2)

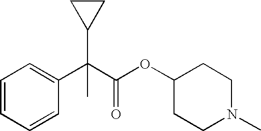

The sub-titled compound was prepared from 2-cyclopropyl-2-phenylpropanoic acid (Isomer 2) (0.16 g) and 4-hydroxy-1-methylpiperidine according to the method of Example 27c. Yield 155 mg.

m/e 288 (M+H)⁺

¹H NMR (400 MHz, DMSO-D₆) δ 7.36-7.31 (m, 4H), 7.26-7.21 (m, 1H), 4.75-4.72 (m, 1H), 2.35-2.20 (m, 2H), 2.20-2.10 (m, 2H), 2.08 (s, 3H), 1.77-1.70 (m, 2H), 1.54-1.43 (m, 3H), 1.17 (s, 3H), 0.62-0.55 (m, 1H), 0.49-0.43 (m, 1H), 0.37-0.26 (m, 2H).

### c) 4-[(2-Cyclopropyl-2-phenylpropanoyl)oxy]-1,1-dimethylpiperidinium iodide (Isomer 2)

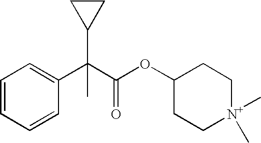

The title compound was prepared from 1-methylpiperidin-4-yl 2-cyclopropyl-2-phenylpropanoate (isomer 2) (147 mg) according to the method of Example 27d. Yield 200 mg.

m/z 302 (M+)⁺

¹H NMR (400 MHz, DMSO-D₆) δ 7.41-7.33 (m, 4H), 7.29-7.26 (m, 1H), 4.96-4.94 (m, 1H), 3.43-3.37 (m, 2H), 3.25-3.13 (m, 2H), 3.07 (s, 3H), 3.05 (s, 3H), 2.12-2.08 (m, 2H), 1.90-1.75 (m, 2H), 1.58-1.53 (m, 1H), 1.20 (s, 3H), 0.61-0.58 (m, 1H), 0.50-0.47 (m, 1H), 0.39-0.30 (m, 2H).

### Pharmacological Analysis

### M₃ Receptor Activity Assay

The affinity (pIC₅₀) of compounds to the M₃ receptor was determined by competition binding of [³H]N-methyl scopolamine (NMS) to CHO-K1 (Chinese Hamster Ovary) cell membranes expressing the human muscarinic acetylcholine M₃ receptor (M₃-ACh) in a scintillation proximity assay (SPA) format.

Membranes were precoupled to SPA beads 5 μg membrane protein per mg of SPA beads, and then incubated at 2 mg/well with serial dilutions of the compounds of the invention, [³H]NMS at 0.2 nM, half Kd (experimentally determined dissociation constant) and assay buffer (20 mM HEPES pH 7.4 containing 5 mM MgCl₂). The assay was conducted in a final volume of 200 μL, in the presence of 1% (v/v) dimethyl sulphoxide (DMSO). Total binding of [³H]NMS was determined in the absence of competing compound and non-specific binding of [3H]NMS was determined in the presence of 1 μM atropine. The plates were incubated for 16 hours at room temperature and then read on Wallac Microbeta™ using a normalised ³H protocol. The pIC₅₀, defined as the negative logarithm of the concentration of compound required for 50% reduction in specific [³H]-NMS, was interpolated empirically between two concentrations of compound where percent inhibition was determined to be greater than and less than 50% respectively. The following table shows the pIC₅₀ figures for a representative selection of compounds:

| Compound of | |
|---|---|
| Example No. | pIC₅₀ |
| 2 | 9.7 |
| 3 | 9.4 |
| 9 | 10.1 |
| 19 | 9.7 |

### Methacholine Induced Bronchoconstriction In Vivo

Dunkin-Hartley guinea-pigs (300-600 g) were supplied by a designated breeding establishment. Animals were dosed with test compound or vehicle either by inhalation in conscious guinea-pigs or by intratracheal instillation (0.5 ml/kg) under recoverable gaseous anaesthesia (5% halothane). Animals were allowed to recover from the anaesthesia prior to the measurement of bronchoconstriction. Up to 48 hours post-dosing guinea-pigs were terminally anaesthetized with sodium pentobarbitone (60 mg/kg), the trachea cannulated for artificial ventilation and the jugular vein was cannulated for intravenous administration of methacholine. The guinea-pigs were ventilated using a constant volume respiratory pump (Harvard Rodent Ventilator model 683) at a rate of 60 breath/min and a tidal volume of 5 ml/kg during surgical preparation. Lung function (lung resistance and compliance) was measured in anaesthetised and ventilated guinea-pigs using a pulmonary measurement Flexivent system (SCIREQ, Montreal, Canada) connected to the tracheal cannulae. The animals were ventilated (quasi-sinusoidal ventilation pattern) at 60 breaths/min at a tidal volume of 5 ml/kg. A positive end expiratory pressure of 2-3 cmH₂O was applied. Respiratory resistance was measured using the Flexivent “snapshot” facility (1 second duration, 1 Hz frequency). Lung resistance and compliance was measured before and after intravenous administration of methacholine (3, 10 and 30 ug/kg). The peak increase in resistance following methacholine challenge was calculated and the effect of the test compound on methacholine-induced lung function changes was calculated.

Percentage inhibition of bronchoconstriction was calculated at each dose of methacholine as follows:

$$\frac{[ \begin{matrix}Change in resistance in vehicle treated group - \\ Change in resistance in compound treated group\end{matrix} ]}{[ Change in resistance in vehicle treated group ]} \times 100$$

Inhibition of Pilocarpine Induced Salivation by i.n. Administered Compounds.

Guinea pigs (450-550 g) supplied by Harlan UK or David Hall, Staffs UK and acclimatised to the in-house facilities for a minimum of three days before use. Guinea pigs were randomly assigned into treatment groups and weighed. Each animal was lightly anaesthetised (4% Halothane) and administered compound or vehicle intranasally (0.5 ml/kg) at up to 24 hours before challenge with pilocarpine. At the test time point, guinea pigs were terminally anaesthetised with urethane (25% solution in H20, 1.5 g/kg). Once sufficient anaesthesia had developed (absence of toe pinch reflex) each animal had an absorbent pad placed in the mouth for 5 minutes to dry residual saliva, this pad was removed and replaced with a new pre-weighed pad for 5 minutes to establish a reading of baseline saliva production. At the end of this 5 minute period the pad was removed and weighed. A new pre-weighed pad was inserted into the mouth before each animal received s.c. pilocarpine administered under the skin at the back of the neck (0.6 mg/kg @ 2 ml/kg). The pad was removed, weighed and replaced with a new pre-weighed pad every 5 minutes up to 15 minutes.

Saliva production was calculated by subtracting the pre-weighed weight of the pad from each 5 minute period post weighed pad and these numbers added together to produce an accumulation of saliva over 15 minutes. Each 5 minute period could be analysed in addition to the whole 15 minute recording period. Baseline production of saliva was assumed to be constant and multiplied by three to produce a reading for baseline saliva production over 15 minutes.

Inhibition of saliva produced by the compound could be calculated by using the following equation: (1−(Test−baseline)/(Veh−baseline))*100.

1. A compound of formula (I): 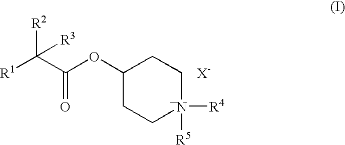
wherein:
R¹ represents phenyl, benzimidazolyl, benzthiazolyl, benzoxazolyl or a 5-6 membered heteroaromatic ring, each of which may be optionally substituted by one or more substituents independently selected from halogen, cyano, nitro, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₅ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
R⁵ represents hydrogen or C₁₋₆ alkyl;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, C₁₋₆ alkoxy, NH₂, NH(C₁₋₆ alkyl) and N(C₁₋₆ alkyl)₂;
or any of R⁷ and R⁸, R⁹ and R¹, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;
and X represents a pharmaceutically acceptable anion of a mono or polyvalent acid.

2. A compound according to claim 1, wherein R¹ represents phenyl, pyridinyl or thienyl which phenyl, pyridinyl or thienyl may be optionally substituted by one or more substituents independently selected from halogen, cyano, hydroxyl, C₁₋₆ alkoxy, SC₁₋₄ alkyl, SO₂C₁₋₄ alkyl, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂.

3. A compound according to claim 1, wherein R² represents a C₃₋₅ cycloalkyl ring which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂.

4. A compound according to claim 1, wherein R³ represents methyl.

5. A compound according to claim 1, wherein R⁴ and R⁵ each independently represent methyl or ethyl.

6. A compound according to claim 1, wherein X represents bromide or iodide.

7. A compound according to claim 1, selected from:
4-{[(2)-2-Cyclopentyl-2-phenylpropanoyl]oxy}-1,1-dimethylpiperidinium X,
4-[(2-Cyclopropyl-2-phenylpropanoyl)oxy]-1,1-dimethylpiperidinium X,
4-[(2-Cyclopentyl-2-pyridin-3-ylpropanoyl)oxy]-1,1-dimethylpiperidinium X,
4-{[2-Cyclopentyl-2-(4-hydroxyphenyl)propanoyl]oxy}-1,1-dimethylpiperidinium X,
4-[(2-Cyclobutyl-2-phenylpropanoyl)oxy]-1,1-dimethylpiperidinium X,
4-{[2-Cyclopentyl-2-(4-methoxyphenyl)propanoyl]oxy}-1,1-dimethylpiperidinium X,
4-{[2-Cyclopentyl-2-(2-thienyl)propanoyl]oxy}-1,1-dimethylpiperidinium X,
4-{[2-Cyclopentyl-2-(5-methyl-2-thienyl)propanoyl]oxy}-1,1-dimethylpiperidinium X,
4-{[2-(3-Bromophenyl)-2-cyclopentylpropanoyl]oxy}-1,1-dimethylpiperidinium X,
4-{[2-(4-Bromophenyl)-2-cyclopentylpropanoyl]oxy}-1,1-dimethylpiperidinium X,
4-{[2-(4-Cyanophenyl)-2-cyclopentylpropanoyl]oxy}-1,1-dimethylpiperidinium X,
4-{[2-(3-Cyanophenyl)-2-cyclopentylpropanoyl]oxy}-1,1-dimethylpiperidinium X,
4-{[2-(3-Methylthiophenyl)-2-cyclopentylpropanoyl]oxy}-1,1-dimethylpiperidinium X,
4-{[2-(4-Methylthiophenyl)-2-cyclopentylpropanoyl]oxy}-1,1-dimethylpiperidinium X,
4-{[2-(4-Methylsulfonylphenyl)-2-cyclopentylpropanoyl]oxy}-1,1-dimethylpiperidinium X,
4-{[2-(3-Methylsulfonylphenyl)-2-cyclopentylpropanoyl]oxy}-1,1-dimethylpiperidinium X,
4-{[2-(4-Fluorophenyl)-2-cyclopentylpropanoyl]oxy}-1,1-dimethylpiperidinium X,
4-{[2-(4-Chlorophenyl)-2-cyclopentylpropanoyl]oxy}-1,1-dimethylpiperidinium X,
4-{[2-(3-Fluorophenyl)-2-cyclopentylpropanoyl]oxy}-1,1-dimethylpiperidinium X, and
4-{[2-(5-Chloro-2-thienyl)-2-cyclobutylpropanoyl]oxy}-1,1-dimethylpiperidinium X
wherein X represents a pharmaceutically acceptable anion of a mono or polyvalent acid.

8. A process for preparing a compound of formula (I) as defined in claim 1, which comprises reacting a compound of formula (IV) wherein R¹, R² and R³ are as defined in formula (I) 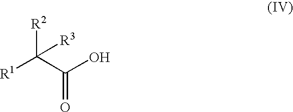
or a C₁₋₆alkyl ester, acid anhydride or acid halide thereof, with a compound of formula (V), wherein R⁴ is as defined in formula (I) 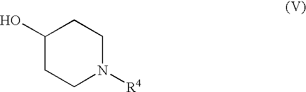
to yield a compound of formula (II) 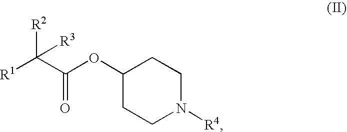
and subsequently reacting (II) with a compound of formula R⁵-Y (III), wherein Y is a leaving group and R⁵ is as defined in formula (I), and optionally carrying out one or more of the following:
converting the compound to a further compound of formula (I),
forming a pharmaceutically acceptable salt with an anion of a mono or polyvalent acid.

9. A pharmaceutical composition comprising a compound of formula (I) as defined in claim 1, in association with a pharmaceutically acceptable adjuvant, diluent or carrier.

10. A process for the preparation of a pharmaceutical composition which comprises mixing a compound of formula (I), as defined in claim 1 with a pharmaceutically acceptable adjuvant, diluent or carrier.

11. (canceled)

12. A method for treatment of chronic obstructive pulmonary disease, which comprises administering to a mammal in need of such treatment a therapeutically effective amount of a compound of formula (I) as claimed in claim 1.

13. A pharmaceutical product comprising, in combination, a first active ingredient which is a compound of formula (I) as defined in claim 1, and a second active ingredient which is selected from;
a phosphodiesterase inhibitor
a β2. adrenoceptor agonist
a modulator of chemokine receptor function
an inhibitor of kinase function
a protease inhibitor
a steroidal glucocorticoid receptor agonist and
a non-steroidal glucocorticoid receptor agonist.

14. A compound of formula (II) 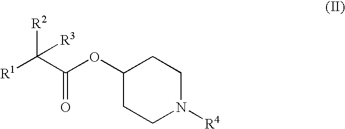
wherein,
R¹ represents phenyl, benzimidazolyl, benzthiazolyl, benzoxazolyl or a 5-6 membered heteroaromatic ring, each of which may be optionally substituted by one or more substituents independently selected from halogen, cyano, nitro, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₅ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, C₁₋₆ alkoxy, NH₂, NH(C₁₋₆ alkyl) and N(C₁₋₆ alkyl)₂;
or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may for a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl.
